(19) United States
(12) Patent Application Publication
(10) Pub. No.: US 2026/0176577 A1
(43) Pub. Date: Jun. 25, 2026

(54) PROSTAGLANDIN E RECEPTOR 4 ANTAGONISTS

(71) Applicant: National Health Research Institutes, Miaoli County (TW)

(72) Inventors: Lun Kelvin Tsou, Miaoli County (TW); Hua-Jung Li, Miaoli County (TW); Guang-Hao Niu, Miaoli County (TW)

(21) Appl. No.: 19/429,222

(22) Filed: Dec. 22, 2025

Related U.S. Application Data
(60) Provisional application No. 63/738,619, filed on Dec. 24, 2024.

(51) Int. Cl. C12N 5/00; C07D 231/32; C07D 403/12; C07D 405/12; C07D 409/12; C12N 5/071; C12N 5/0775; C12N 5/0797

(52) U.S. Cl. CPC C12N 5/0018; C07D 231/32; C07D 403/12; C07D 405/12; C07D 409/12; C12N 5/0623; C12N 5/0631; C12N 5/0662; C12N 2501/999

(57) ABSTRACT

The compounds of Formula (I):

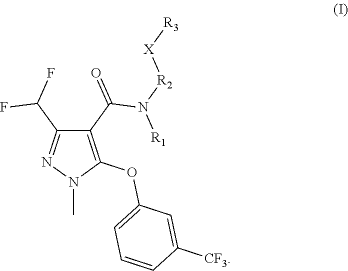

Each variable is defined. Also disclosed is method of producing secresomes enriched for proteins or mRNAs using such compounds.

### CROSS REFERENCES TO RELATED APPLICATION

This application claims the benefit of and priority to U.S. Application Ser. No. 63/738,619 filed on Dec. 24, 2024, the content and disclosure of which are incorporated herein by reference in its entirety.

### BACKGROUND

Secresomes are biological factors including functional proteins and nucleic acids that are released by living cells into their extracellular space.

They provide potential therapies in a variety of pathologies. Secresomes of mesenchymal stem cells (MSC) have been found useful in treating central nervous system (CNS) conditions such as traumatic brain injury, stroke, cerebral infarction, Alzheimer's disease, and Parkinson's disease. See Chen et al., Stem Cells Translational Medicine 8, 707-723 (2019). It has been demonstrated that MSC-derived secresomes suppress astrogliosis and inflammation, increase blood-brain barrier integrity in damaged hippocampi, and repair deficiencies of cognition, memory, and learning caused by damage in hippocampus CA1.

Secresomes are also important in cancer progression, making them useful in cancer diagnosis and prognosis, and development of new cancer therapies when engineered to contain anti-cancer factors.

A prostaglandin E receptor 4 (EP4) antagonist can induce release of secresomes from stem cells. See Hua-Jung Li, U.S. Pat. No. 11,149,277 B2. The released secresomes are enriched and isolated for treating spinal cord trauma, learning disabilities, myocardial infarction, muscular dystrophy, baldness, deafness, vision loss, diabetes, or wound.

Current EP4 antagonists induce release of secresomes only in very limited amounts. Induced release of specific, targeted proteins remains unachievable.

There is a need to develop new EP4 antagonists that can effectively promote release of secresomes including specific therapeutic proteins and nucleic acids.

### SUMMARY OF THE INVENTION

It is unexpectedly found that certain pyrazole compounds can effectively induce release of secresomes as useful therapeutic agents.

Accordingly, one aspect of the invention relates to compounds of Formula (I):

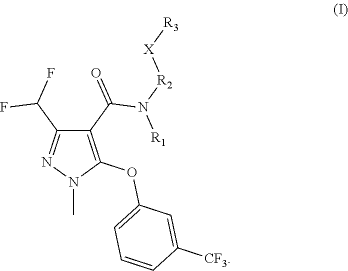

In this formula, R₁ is H or NH₂, or R₁ and R₂, together with the nitrogen atom they bond to, form a heterocycloalkyl ring (e.g., 4- to 8-membered heterocycloalkyl and 5- to 6-membered heterocycloalkyl); R₂ is a bond, CH₂, (CH₂)₂, (CH₂)₃, (CH₂)₄, (CH₂)₅, (CH₂)₄CH(COOH), or (CH₂)₆; X is a bond, C(═NH), NH, O, NR₃′, N(NH₂), NHC(O), or NHC(S), R₃′ being C₁-C₆ alkyl; and R₃ is phosphonate, phosphonic, carboxylic, C₃-C₆ alkyl, C₁-C₆ alkoxy, C₃-C₁₀ cycloalkyl, 3- to 10-membered heterocycloalkyl, amino, aryl, or heteroaryl.

Compounds of Formula (I) can have one or any combination of the following features:
(i) R₁ is H;
(ii) each of R₂ and X is a bond;
(iii) R₃ is aryl, heteroaryl, cycloalkyl, or heterocycloalkyl; and each of aryl, heteroaryl, cycloalkyl, and heterocycloalkyl is substituted or unsubstituted;
(iv) R₃ is aryl or heteroaryl substituted with heterocycloalkyl, heteroaryl, amido, trifluoromethyl, alkoxy, carboxylate, or carboxamide;
(v) R₃ is

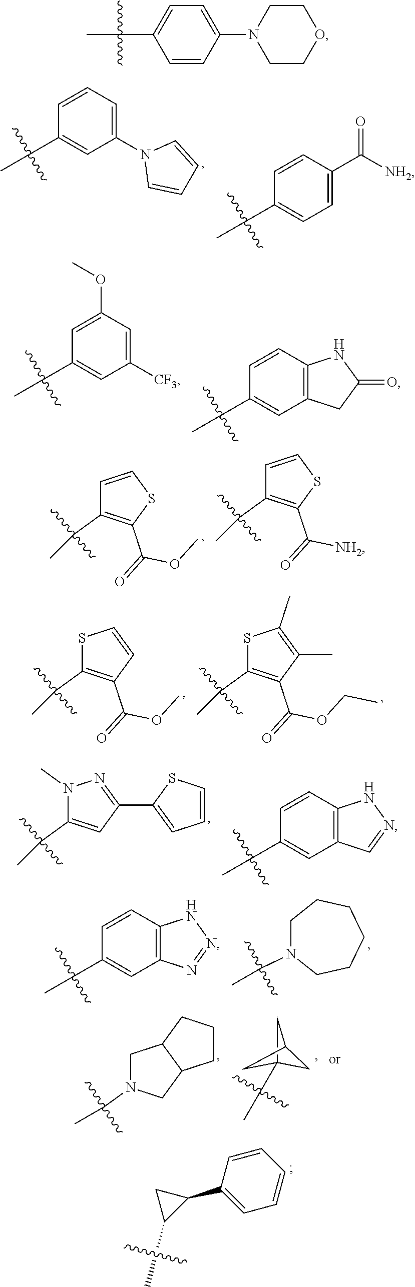

(vi) R₁ is H; R₂ is a bond, CH₂, (CH₂)₂, (CH₂)₃, (CH₂)₄, (CH₂)₅, or (CH₂)₆;
(vii) X is a bond;
(viii) R₃ is aryl, heteroaryl, alkoxy, cycloalkyl, heterocycloalkyl, amino, phosphonate, or C₂-C₆ alcohol;
(ix) R₃ is unsubstituted or substituted with halo, OH, NH₂, alkyl, carboxylate, alkoxy, sulfonamide, or carboxyl;
(x) R₂—X—R₃ is

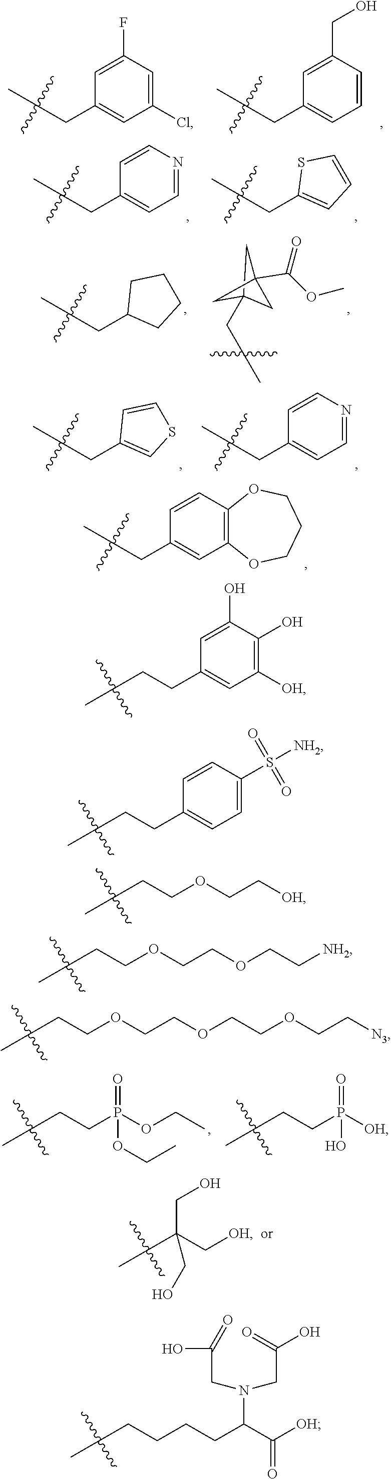

(xi) R₁ is H; R₂ is a bond; X is NH, N(NH₂), NHC(O), or NHC(S); and R₃ is amino, C₃-C₆ alkyl, C₁-C₆ alkoxy, C₃-C₁₀ cycloalkyl, 3- to 10-membered heterocycloalkyl, aryl, or heteroaryl;
(xii) X—R₃ is

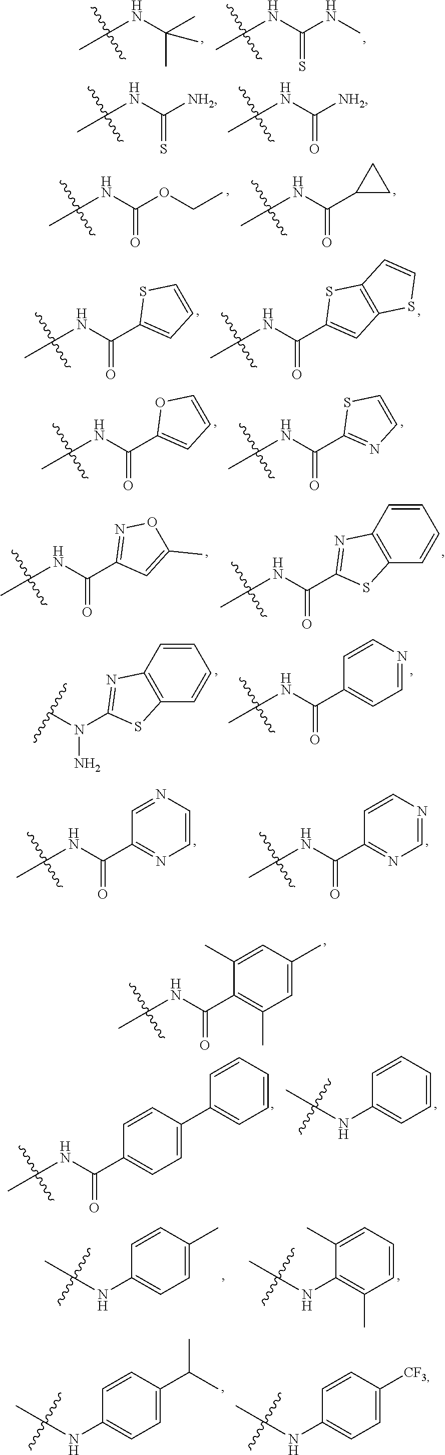

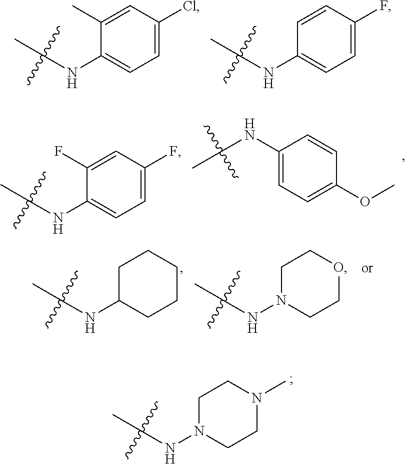

(xiii) R₁ and R₂, together with the nitrogen atom they bond to, form a heterocycloalkyl ring;
X is a bond, C(═NH), NH, N(NH₂), NHC(O), or NHC(S); and
R₃ is amino, C₃-C₆ alkyl, C₃-C₁₀ cycloalkyl, 3- to 10-membered heterocycloalkyl, aryl, or heteroaryl;
(xiv) N(R₁)—R₂—X—R₃ is

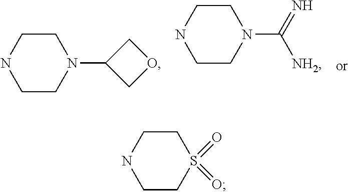

(xv) R₂ is a bond, and X is C(═NH), NH, N(NH₂), NHC(O), or NHC(S); and
(xvi) each of R₂ and X is a bond, R₃ is aryl, heteroaryl, cycloalkyl, or heterocycloalkyl; and each of aryl, heteroaryl, cycloalkyl, and heterocycloalkyl is substituted or unsubstituted;

Other features include:
(i) R₂ is CR₂ₐR_{2b}, each of R₂ₐ and R_{2b} independently is H, CH₃, or cyclopropyl; and
(ii) R₃ is phenyl substituted with one, two, or three chemical groups selected from COOH, F, Cl, CH₃, and OCH₃.

Exemplary compounds, Compounds 1-67, of this invention are shown in Table 1 below. Preferred compounds are Compounds 12, 28, 35, 45, and 65.

TABLE 1

| | |
|---|---|
| 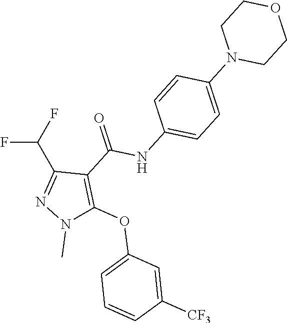 [SMILES: Cn1nc(C(F)F)c(C(=O)Nc2ccc(N3CCOCC3)cc2)c1Oc1cccc(C(F)(F)F)c1] | 1 |
| 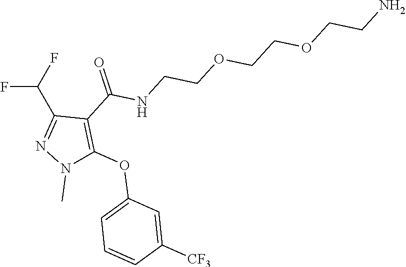 [SMILES: Cn1nc(C(F)F)c(C(=O)NCCOCCOCCN)c1Oc1cccc(C(F)(F)F)c1] | 2 |
| 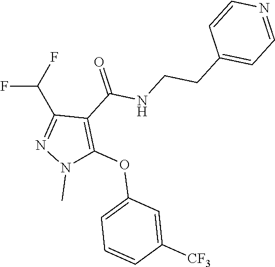 [SMILES: Cn1nc(C(F)F)c(C(=O)NCCc2ccncc2)c1Oc1cccc(C(F)(F)F)c1] | 3 |
| 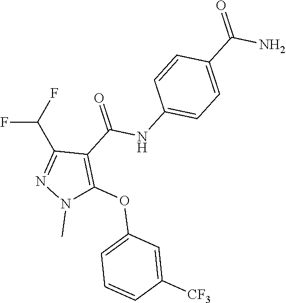 [SMILES: Cn1nc(C(F)F)c(C(=O)Nc2ccc(C(N)=O)cc2)c1Oc1cccc(C(F)(F)F)c1] | 4 |
| 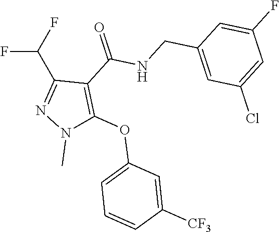 [SMILES: Cn1nc(C(F)F)c(C(=O)NCc2cc(F)cc(Cl)c2)c1Oc1cccc(C(F)(F)F)c1] | 5 |
| 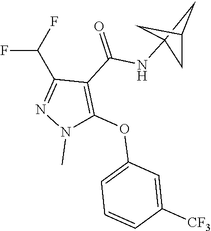 [SMILES: Cn1nc(C(F)F)c(C(=O)NC23CC(C2)C3)c1Oc1cccc(C(F)(F)F)c1] | 6 |
| 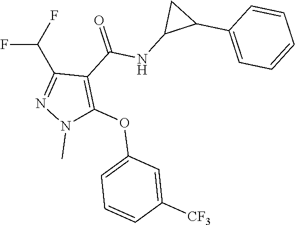 [SMILES: Cn1nc(C(F)F)c(C(=O)NC2CC2c2ccccc2)c1Oc1cccc(C(F)(F)F)c1] | 7 |
| 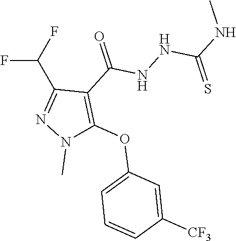 [SMILES: CNC(=S)NNC(=O)c1c(C(F)F)nn(C)c1Oc1cccc(C(F)(F)F)c1] | 8 |
| 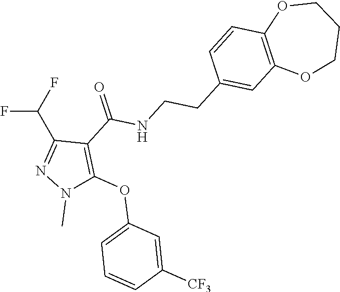 [SMILES: Cn1nc(C(F)F)c(C(=O)NCCc2ccc3c(c2)OCCCO3)c1Oc1cccc(C(F)(F)F)c1] | 9 |
| 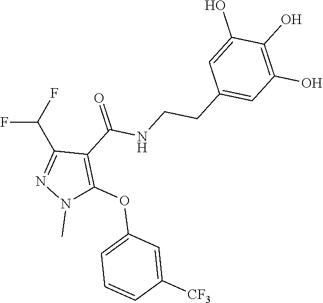 [SMILES: Cn1nc(C(F)F)c(C(=O)NCCc2cc(O)c(O)c(O)c2)c1Oc1cccc(C(F)(F)F)c1] | 10 |
| 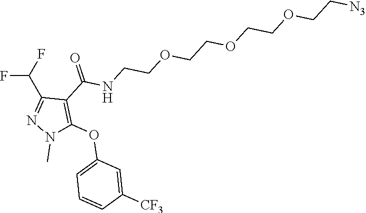 [SMILES: Cn1nc(C(F)F)c(C(=O)NCCOCCOCCOCCN=[N+]=[N-])c1Oc1cccc(C(F)(F)F)c1] | 11 |
| 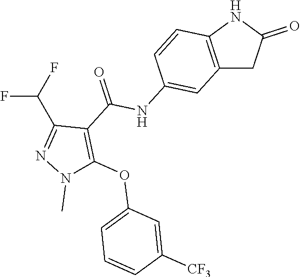 [SMILES: Cn1nc(C(F)F)c(C(=O)Nc2ccc3c(c2)CC(=O)N3)c1Oc1cccc(C(F)(F)F)c1] | 12 |
| 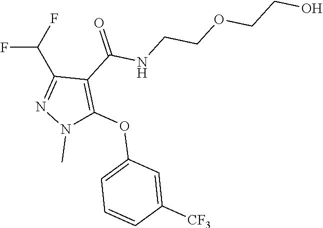 [SMILES: Cn1nc(C(F)F)c(C(=O)NCCOCCO)c1Oc1cccc(C(F)(F)F)c1] | 13 |
| 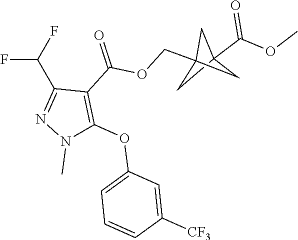 [SMILES: COC(=O)C12CC(COC(=O)c3c(C(F)F)nn(C)c3Oc3cccc(C(F)(F)F)c3)(C1)C2] | 14 |
| 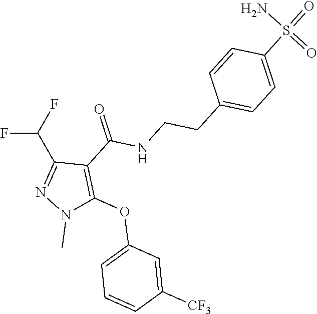 [SMILES: Cn1nc(C(F)F)c(C(=O)NCCc2ccc(S(N)(=O)=O)cc2)c1Oc1cccc(C(F)(F)F)c1] | 15 |
| 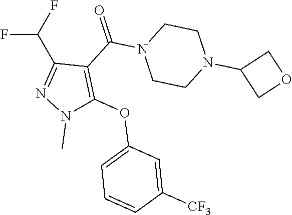 [SMILES: Cn1nc(C(F)F)c(C(=O)N2CCN(C3COC3)CC2)c1Oc1cccc(C(F)(F)F)c1] | 16 |
| 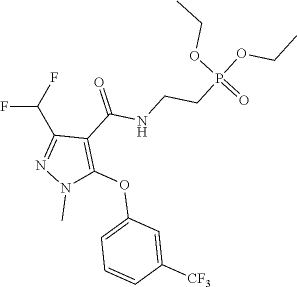 [SMILES: CCOP(=O)(CCNC(=O)c1c(C(F)F)nn(C)c1Oc1cccc(C(F)(F)F)c1)OCC] | 17 |
| 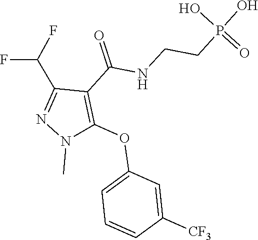 [SMILES: Cn1nc(C(F)F)c(C(=O)NCCP(=O)(O)O)c1Oc1cccc(C(F)(F)F)c1] | 18 |
| 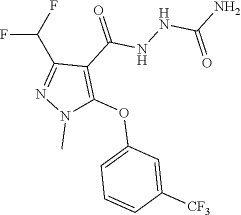 [SMILES: Cn1nc(C(F)F)c(C(=O)NNC(N)=O)c1Oc1cccc(C(F)(F)F)c1] | 19 |
| 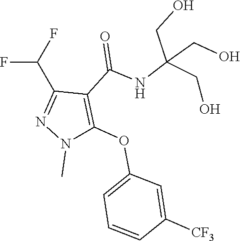 [SMILES: Cn1nc(C(F)F)c(C(=O)NC(CO)(CO)CO)c1Oc1cccc(C(F)(F)F)c1] | 20 |
| 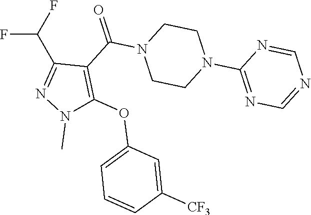 [SMILES: Cn1nc(C(F)F)c(C(=O)N2CCN(c3ncncn3)CC2)c1Oc1cccc(C(F)(F)F)c1] | 21 |
| 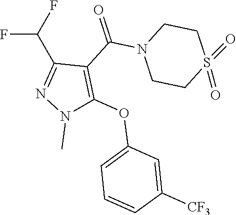 [SMILES: Cn1nc(C(F)F)c(C(=O)N2CCS(=O)(=O)CC2)c1Oc1cccc(C(F)(F)F)c1] | 22 |
| 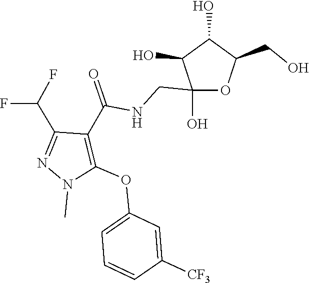 [SMILES: Cn1nc(C(F)F)c(C(=O)NCC2(O)O[C@H](CO)[C@@H](O)[C@@H]2O)c1Oc1cccc(C(F)(F)F)c1] | 23 |
| 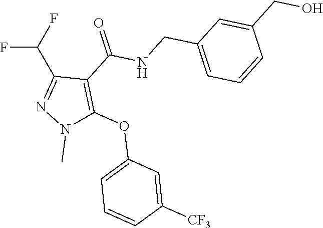 [SMILES: Cn1nc(C(F)F)c(C(=O)NCc2cccc(CO)c2)c1Oc1cccc(C(F)(F)F)c1] | 24 |
| 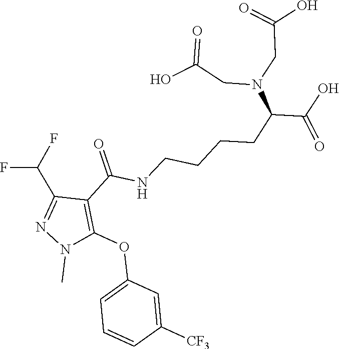 [SMILES: Cn1nc(C(F)F)c(C(=O)NCCCC[C@H](C(=O)O)N(CC(=O)O)CC(=O)O)c1Oc1cccc(C(F)(F)F)c1] | 25 |
| 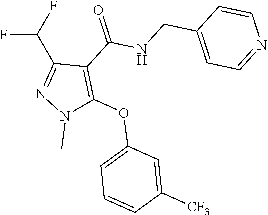 [SMILES: Cn1nc(C(F)F)c(C(=O)NCc2ccncc2)c1Oc1cccc(C(F)(F)F)c1] | 26 |
| 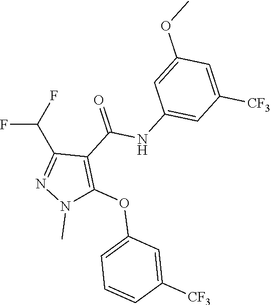 [SMILES: COc1cc(NC(=O)c2c(C(F)F)nn(C)c2Oc2cccc(C(F)(F)F)c2)cc(C(F)(F)F)c1] | 27 |
| 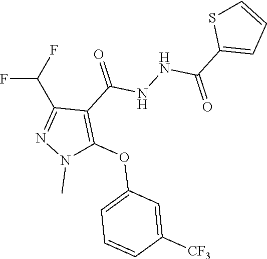 [SMILES: Cn1nc(C(F)F)c(C(=O)NNC(=O)c2cccs2)c1Oc1cccc(C(F)(F)F)c1] | 28 |
| 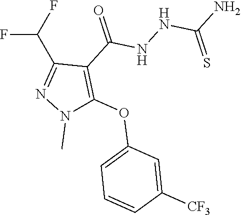 [SMILES: Cn1nc(C(F)F)c(C(=O)NNC(N)=S)c1Oc1cccc(C(F)(F)F)c1] | 29 |
| 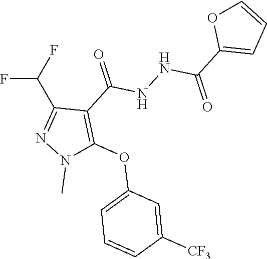 [SMILES: Cn1nc(C(F)F)c(C(=O)NNC(=O)c2ccco2)c1Oc1cccc(C(F)(F)F)c1] | 30 |
| 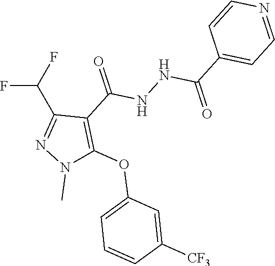 [SMILES: Cn1nc(C(F)F)c(C(=O)NNC(=O)c2ccncc2)c1Oc1cccc(C(F)(F)F)c1] | 31 |
| 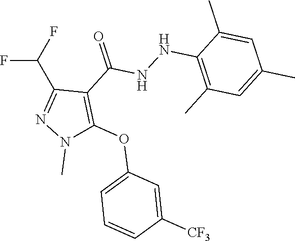 [SMILES: Cc1cc(C)c(NNC(=O)c2c(C(F)F)nn(C)c2Oc2cccc(C(F)(F)F)c2)c(C)c1] | 32 |
| 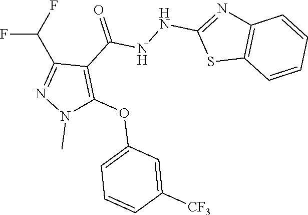 [SMILES: Cn1nc(C(F)F)c(C(=O)NNc2nc3ccccc3s2)c1Oc1cccc(C(F)(F)F)c1] | 33 |
| 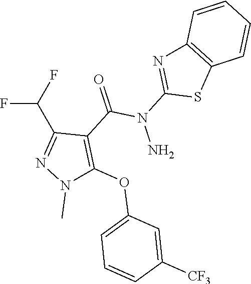 [SMILES: Cn1nc(C(F)F)c(C(=O)N(N)c2nc3ccccc3s2)c1Oc1cccc(C(F)(F)F)c1] | 34 |
| 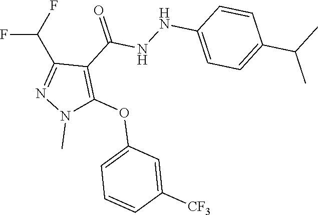 [SMILES: CC(C)c1ccc(NNC(=O)c2c(C(F)F)nn(C)c2Oc2cccc(C(F)(F)F)c2)cc1] | 35 |
| 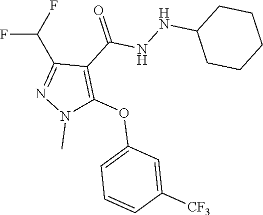 [SMILES: Cn1nc(C(F)F)c(C(=O)NNC2CCCCC2)c1Oc1cccc(C(F)(F)F)c1] | 36 |
| 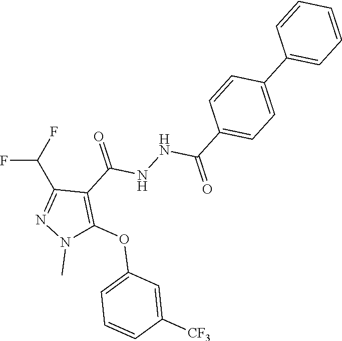 [SMILES: Cn1nc(C(F)F)c(C(=O)NNC(=O)c2ccc(-c3ccccc3)cc2)c1Oc1cccc(C(F)(F)F)c1] | 37 |
| 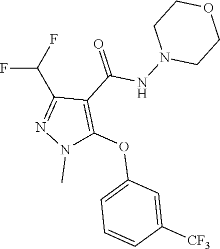 [SMILES: Cn1nc(C(F)F)c(C(=O)NN2CCOCC2)c1Oc1cccc(C(F)(F)F)c1] | 38 |
| 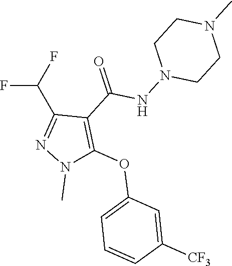 [SMILES: CN1CCN(NC(=O)c2c(C(F)F)nn(C)c2Oc2cccc(C(F)(F)F)c2)CC1] | 39 |
| 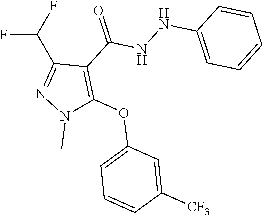 [SMILES: Cn1nc(C(F)F)c(C(=O)NNc2ccccc2)c1Oc1cccc(C(F)(F)F)c1] | 40 |
| 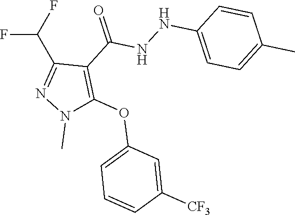 [SMILES: Cc1ccc(NNC(=O)c2c(C(F)F)nn(C)c2Oc2cccc(C(F)(F)F)c2)cc1] | 41 |
| 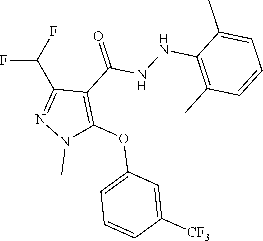 [SMILES: Cc1cccc(C)c1NNC(=O)c1c(C(F)F)nn(C)c1Oc1cccc(C(F)(F)F)c1] | 42 |
| 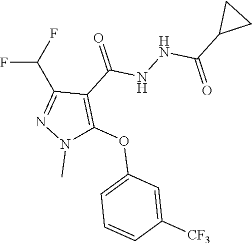 [SMILES: Cn1nc(C(F)F)c(C(=O)NNC(=O)C2CC2)c1Oc1cccc(C(F)(F)F)c1] | 43 |
| 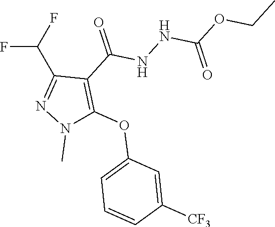 [SMILES: CCOC(=O)NNC(=O)c1c(C(F)F)nn(C)c1Oc1cccc(C(F)(F)F)c1] | 44 |
| 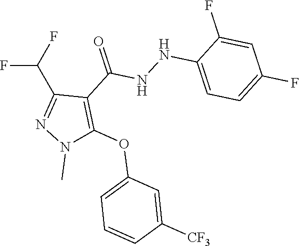 [SMILES: Cn1nc(C(F)F)c(C(=O)NNc2ccc(F)cc2F)c1Oc1cccc(C(F)(F)F)c1] | 45 |
| 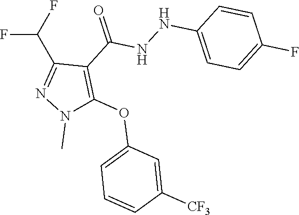 [SMILES: Cn1nc(C(F)F)c(C(=O)NNc2ccc(F)cc2)c1Oc1cccc(C(F)(F)F)c1] | 46 |
| 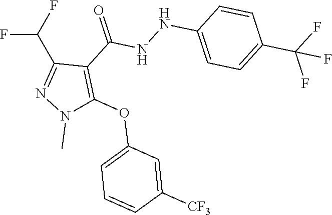 [SMILES: Cn1nc(C(F)F)c(C(=O)NNc2ccc(C(F)(F)F)cc2)c1Oc1cccc(C(F)(F)F)c1] | 47 |
| 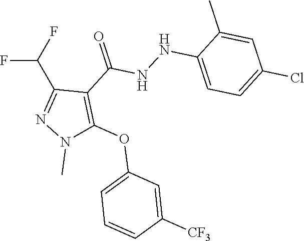 [SMILES: Cc1cc(Cl)ccc1NNC(=O)c1c(C(F)F)nn(C)c1Oc1cccc(C(F)(F)F)c1] | 48 |
| 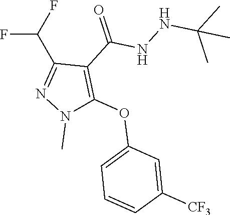 [SMILES: Cn1nc(C(F)F)c(C(=O)NNC(C)(C)C)c1Oc1cccc(C(F)(F)F)c1] | 49 |
| 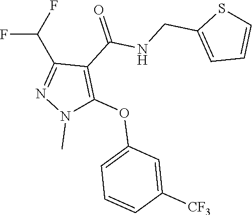 [SMILES: Cn1nc(C(F)F)c(C(=O)NCc2cccs2)c1Oc1cccc(C(F)(F)F)c1] | 50 |
| 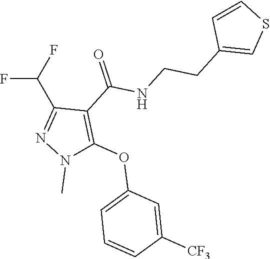 [SMILES: Cn1nc(C(F)F)c(C(=O)NCCc2ccsc2)c1Oc1cccc(C(F)(F)F)c1] | 51 |
| 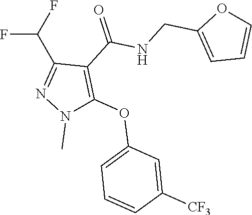 [SMILES: Cn1nc(C(F)F)c(C(=O)NCc2ccco2)c1Oc1cccc(C(F)(F)F)c1] | 52 |
| 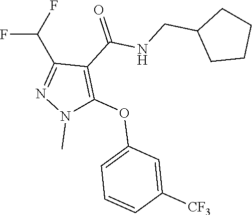 [SMILES: Cn1nc(C(F)F)c(C(=O)NCC2CCCC2)c1Oc1cccc(C(F)(F)F)c1] | 53 |
| 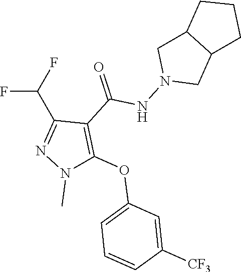 [SMILES: Cn1nc(C(F)F)c(C(=O)NN2CC3CCCC3C2)c1Oc1cccc(C(F)(F)F)c1] | 54 |
| 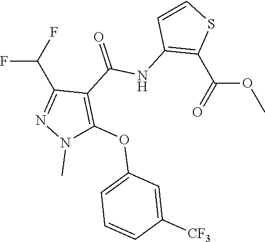 [SMILES: COC(=O)c1sccc1NC(=O)c1c(C(F)F)nn(C)c1Oc1cccc(C(F)(F)F)c1] | 55 |
| 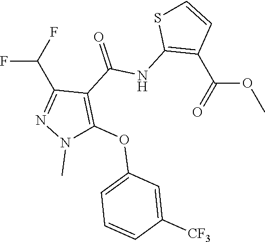 [SMILES: COC(=O)c1ccsc1NC(=O)c1c(C(F)F)nn(C)c1Oc1cccc(C(F)(F)F)c1] | 56 |
| 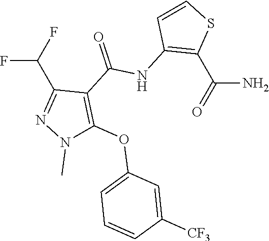 [SMILES: Cn1nc(C(F)F)c(C(=O)Nc2ccsc2C(N)=O)c1Oc1cccc(C(F)(F)F)c1] | 57 |
| 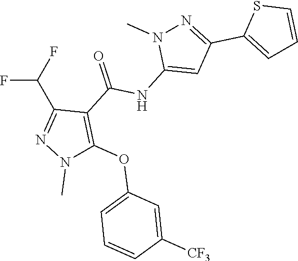 [SMILES: Cn1nc(-c2cccs2)cc1NC(=O)c1c(C(F)F)nn(C)c1Oc1cccc(C(F)(F)F)c1] | 58 |
| 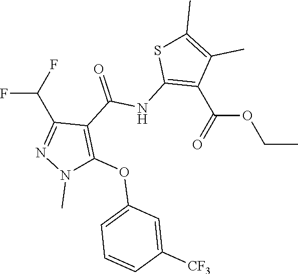 [SMILES: CCOC(=O)c1c(NC(=O)c2c(C(F)F)nn(C)c2Oc2cccc(C(F)(F)F)c2)sc(C)c1C] | 59 |
| 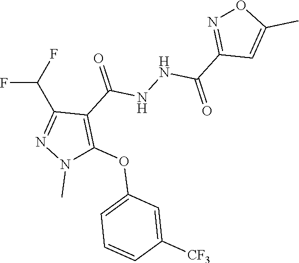 [SMILES: Cc1cc(C(=O)NNC(=O)c2c(C(F)F)nn(C)c2Oc2cccc(C(F)(F)F)c2)no1] | 60 |
| 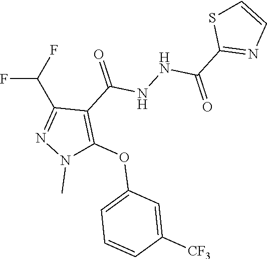 [SMILES: Cn1nc(C(F)F)c(C(=O)NNC(=O)c2nccs2)c1Oc1cccc(C(F)(F)F)c1] | 61 |
| 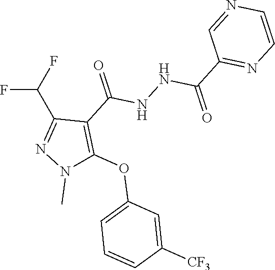 [SMILES: Cn1nc(C(F)F)c(C(=O)NNC(=O)c2cnccn2)c1Oc1cccc(C(F)(F)F)c1] | 62 |
| 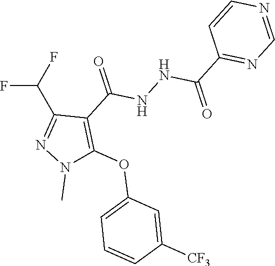 [SMILES: Cn1nc(C(F)F)c(C(=O)NNC(=O)c2ccncn2)c1Oc1cccc(C(F)(F)F)c1] | 63 |
| 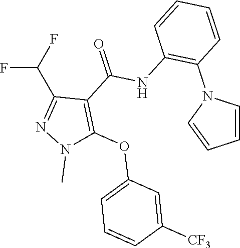 [SMILES: Cn1nc(C(F)F)c(C(=O)Nc2ccccc2-n2cccc2)c1Oc1cccc(C(F)(F)F)c1] | 64 |
| 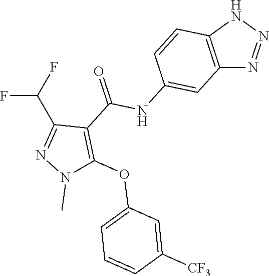 [SMILES: Cn1nc(C(F)F)c(C(=O)Nc2ccc3[nH]nnc3c2)c1Oc1cccc(C(F)(F)F)c1] | 65 |
| 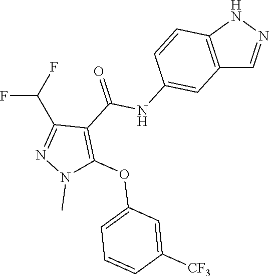 [SMILES: Cn1nc(C(F)F)c(C(=O)Nc2ccc3[nH]ncc3c2)c1Oc1cccc(C(F)(F)F)c1] | 66 |
| 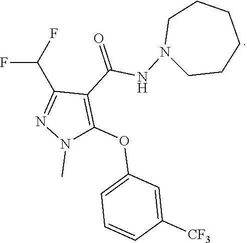 [SMILES: Cn1nc(C(F)F)c(C(=O)NN2CCCCCC2)c1Oc1cccc(C(F)(F)F)c1] | 67 |

Another aspect of this invention relates to a method of producing secresomes enriched with proteins and mRNAs, the method comprising: (i) obtaining an isolated population of stem cells, wherein the stem cells are mesenchymal stem cells, mammary epithelial stem cells, neural stem cells or cancer stem cells, (ii) culturing the isolated population of stem cells in a culture medium suitable for culturing the stem cells, wherein the culture medium contains an amount of any one of the compounds described above, the amount being effective for inducing release of secresomes enriched for proteins and miRNAs for maintenance of stem cell properties or for cell regeneration from the stem cells, for a sufficient period of time to allow release of the secresomes, whereby the secresomes are released from the stem cells into the culture medium, and isolating the secresomes from the culture medium, and have a higher protein and miRNA content, as compared to control stem cells not treated with compounds. The secresomes are useful for maintenance of stem cell properties or for cell regeneration. They are also suitable therapeutic agents for treating conditions including central nervous system (CNS) conditions such as traumatic brain injury, stroke, cerebral infarction, Alzheimer's disease, Parkinson's disease, astrogliosis and inflammation, damaged hippocampi, deficiencies of cognition, memory, and learning caused by damage in hippocampus CA1, cancer treatment or diagnosis, spinal cord trauma, myocardial infarction, muscular dystrophy, baldness, deafness, vision loss, diabetes, and wound.

In a preferred method, the stem cells are cultured for 4 to 8 days. The effective amount of the compound is typically in the range of 1-40 μg/ml.

Suitable stem cells include mesenchymal stem cells, mammary epithelial stem cells, neural stem cells, and the like.

The isolating step can include collecting the culture medium containing the secresomes released from the stem cells.

Also within the scope of this invention are pharmaceutical compositions containing a pharmaceutically acceptable carrier and a secresome prepared above.

Still within the scope of the invention is a method of treating a condition using the secresome or pharmaceutical composition described above. The current invention further includes use of such a secresome or composition for treating a condition or for the manufacture of a medicament for treating the condition.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “amino” refers to a radical derived from amine, which is unsubstituted (NH₂) or mono-/di-substituted with alkyl, aryl, cycloalkyl, heterocycloalkyl, or heteroaryl. The term “alkyl-amino” refers to an amino radical represented by alkyl-NH—. The termi “dialkylamino” refers to an amino radical connecting directly to two alkyl groups. The term “arylamino” refers to aryl-NH—. The term “diarylamino” refers to an amino radical connecting directly to two aryl groups. The term “aminoalkyl” refers to NR¹R²-alkyl, i.e., an alkyl that is substituted with at least one amino group, in which each of R¹ and R², independently, is hydrogen, a C₁₋₇ alkyl group, a C₃₋₂₀ heterocycloalkyl group, a C₆₋₂₀ aryl group, alkylaryl, or a 5-20 membered heteroaryl group. Examples of aminoalkyl include aminomethyl and 2-aminoethyl.

The term “alkyl” herein refers to a straight or branched hydrocarbon group, containing 1-20 (e.g., 1-10 and 1-6) carbon atoms. Examples include methyl, ethyl, n-propyl, i-propyl, n-butyl, i-butyl, and t-butyl. The term “haloalkyl” refers to alkyl substituted with one or more halogen (chloro, fluoro, bromo, or iodo) atoms. Examples include trifluoromethyl, bromomethyl, and 4,4,4-trifluorobutyl.

The term “cycloalkyl” refers to a saturated and partially unsaturated (i.e., nonaromatic) monocyclic, bicyclic, tricyclic, or tetracyclic hydrocarbon group having 3 to 12 carbons. Examples include cyclopropyl, cyclobutyl, cyclopentyl, cyclopentenyl, cyclohexyl, cyclohexenyl, cycloheptyl, cyclooctyl, indane, and indene.

The term “heterocycloalkyl” refers to a nonaromatic 5-8 membered monocyclic, 8-12 membered bicyclic, or 11-14 membered tricyclic ring system having one or more heteroatoms (e.g., O, N, P, and S). Examples include piperazinyl, imidazolidinyl, azepanyl, pyrrolidinyl, dihydrothia-diazolyl, dioxanyl, morpholinyl, 1,1-dioxidothiomorpholinyl, hexahydrocyclopenta[c]pyrrol-2(1H)-yl, tetrahydropuranyl, and tetrahydrofuranyl.

The term “aryl” refers to a 6-carbon monocyclic, 10-carbon bicyclic, 14-carbon tricyclic aromatic ring system. Examples of aryl groups include phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyrazinyl, pyridyl, furyl, imidazolyl, benzimidazolyl, pyrimidinyl, thienyl, quinolinyl, indolyl, indolinyl, indaxolyl, thiazolyl, thienothiophenyl, and benzothiazolyl.

Alkyl, amino, cycloalkyl, heterocycloalkyl, aryl, and heteroaryl mentioned herein include both substituted and unsubstituted moieties. Possible substituents on amino, alkylthio, cycloalkyl, cycloalkylaryl, heterocycloalkyl, heterocycloalkylaryl, aralkyl, heteroaralkyl, alkoxy, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, sulfamoyl (—SO₂NH₂), C₁-C₁₀ alkylsulfonamino, arylsulfonamino, C₁-C₁₀ alkylimino, arylimino, hydroxyl, halo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, halo, hydroxyl, cyano, nitro, nitroso, azido (N₃), acyl, thioacyl, and acyloxy. On the other hand, possible substituents on alkyl or alkenyl include all of the above-recited substituents except C₁-C₁₀ alkyl. Cycloalkyl, heterocycloalkyl, aryl, and heteroaryl can also be fused with each other such as 3,4-dihydro-2H-1,5-benzodioxepin-7-yl, 2-oxo-2,3-dihydro-1H-indol-5-yl.

Exemplary substituents include F, Cl, OH, CH₃, CH₂F, CHF₂, CF₃, —CH₂CH₃, —CH(CH₃)₂, —CH₂OH, —COOH, —OCH₃, —C(O)NH₂, —C(O)OCH₃, —C(O)OCH₂CH₃, —SO₂NH₂, —P(O)(OH)₂, —P(O)(OCH₂CH₃)₂, —O—CH₂CH₂NH₂, —O—CH₂CH₂OCH₂CH₂N₃, phenyl,

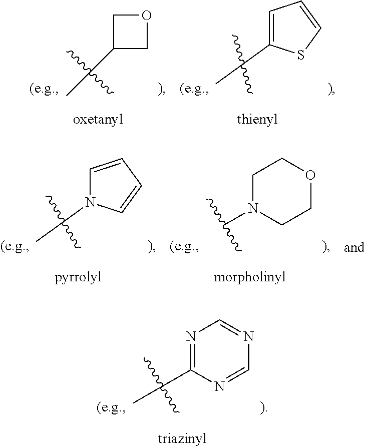 [SMILES: *C1COC1 ; *c1cccs1 ; *n1cccc1 ; *N1CCOCC1 ; *c1ncncn1]

The term “halo” or “halogen” herein refers to a fluoro, chloro, bromo, or iodo radical. Examples include a fluoro radical (F), chloro radical (Cl), and a bromo radical (Br).

The term “cyano” refers to the “CN” radical. The term “nitro” refers to the “NO₂” radical. The term “nitroso” refers to the “NO” radical. The term “azido” refers to —N₃.

The term “alkylene” refers to a straight or branched hydrocarbon group, containing 1-20 carbon atoms (e.g., C₁₋₆ and C₁₋₃) and two monovalent radical centers or a bivalent radical center derived by the removal of two hydrogen atoms from one or more carbon atoms of a parent alkane. Exemplary alkylene groups include, but not limited to —CH₂—, —CH₂CH₂—, —CH(CH₃)—, —C(CH₃)₂—, —CH₂CH₂CH₂—, —CH(CH₃)CH₂—.

The term “alkenyl”, as employed herein as such or as part of another group, refers to an aliphatic hydrocarbon group having at least 2 carbon atoms and containing one or several double bonds. Representative examples include, but are not limited to, ethenyl, propenyl and butenyl.

The term “cycloalkenyl” refers to a partially unsaturated (i.e., nonaromatic) monocyclic, bicyclic, tricyclic, or tetracyclic hydrocarbon having 3 to 12 carbon atoms.

The term “heterocycloalkenyl” refers to a partially unsaturated (i.e., nonaromatic) 5-8 membered monocyclic, 8-12 membered bicyclic, or 11-14 membered tricyclic ring system having one or more heteroatoms (e.g., O, N, P, and S).

The term “alkylsulfonamino” refers to alkyl-S(═O)N(R¹)— group, wherein R¹ is hydrogen, a C₁₋₇ alkyl group, a C₃₋₂₀ heterocycloalkyl group, a C₆₋₂₀ aryl group, alkylaryl, or a 5-20 membered heteroaryl group. The term “arylsulfonamino” refers to aryl-S(═O)N(R¹)—, R¹ is define as above.

The term “alkylimino” refers to —CR¹═N(alkyl). The term “arylamino” refers to —CR¹═N(alkyl). R¹ is defined above.

The term “acylamino” or “amido” refers to R²—C(O)—N(R¹)—. The term “aminoacyl” refers to N(R¹)(R²)C(O)—, in which each of R¹ and R², independently, is hydrogen, a C₁₋₇ alkyl group, a C₃₋₂₀ heterocycloalkyl group, a C₆₋₂₀ aryl group, alkylaryl, or a 5-20 membered heteroaryl group.

The term “thio” includes —SH, alkyl-S— (alkylthio), and aryl-S— (arylthiol).

The term “alkylsulfonyl” refers to alkyl-S(O)₂—. The term “arylsulfonyl” refers to aryl-S(O)₂—.

The term “aminothioacyl” refers to N(R¹)(R²)C(S)—. The term “amidino” refers to R¹C(═NR²)N(R³))—, in which R¹ and R² are defined above and R³ is hydrogen, a C₁₋₇ alkyl group, a C₃₋₂₀ heterocycloalkyl group, a C₆₋₂₀ aryl group, alkylaryl, or a 5-20 membered heteroaryl group.

The term “ureido” refers to R¹R²NC(O)NR³—. The term “thioureido” refers to R¹R²NC(S)NR³—. R¹, R², and R³ are defined above.

The term “phosphonate” or “phosphonic” refers to —P(O)(OR¹)(OR²). R¹ and R² are defined above. In phosphonic, both R¹ and R² are H.

The term “acyl” refers to R¹C(O)—. The term “thioacyl” refers to refers to R¹C(S)—. The term “acyloxy” or “carboxyl” refers to R¹COO—.

The term “alkynyl”, as employed herein as such or as part of another group, refers to an aliphatic hydrocarbon group having at least 2 carbon atoms and containing one or several triple bonds. Representative examples include, but are not limited to, ethynyl, propynyl and butynyl.

The term “alkoxy” refers to an —O-alkyl group. Representative examples of C₁₋₇ alkoxy include, but are not limited to methoxy, ethoxy, propoxy, butoxy, isobutoxy, sec-butoxy and Ze/7-butoxy. Alkoxy also includes haloalkoxy, namely, alkoxy substituted with one or more halogens, e.g., —O—CH₂Cl, —O—CF₃ and —O—CHClCH₂C₁.

The term “alkylthio” refers to an —S-alkyl group. Representative examples of C₁₋₇ alkylthio include, but are not limited to methylthio, ethylthio, propylthio, butylthio, isobutylthio, sec-butylthio and Ze/7-butylthio. Alkylthio also includes haloalkylthio, namely, alkylthio substituted with one or more halogens, e.g., —S—CH₂Cl, —S—CF₃ and —S—CHClCH₂Cl.

The term “oxo”, as employed herein as such or as part of another group, refers to oxygen atom linked to another atom by a double bond (=O). The term “carbonyl” refers to —C(O)—. The term “carboxyl” refers to —COOH.

The term “aralkyl” refers to alkyl substituted with an aryl group. Examples include benzyl and naphthylmethyl. The term “aryloxy” refers to aryl-O—.

The term “heteroaryloxy” refers to heteroaryl-O—.

The term “cycloalkylaryl” refers to a cycloalkyl ring fused with aryl. The term “heterocycloalkylaryl” refers to a heterocycloalkyl ring fused with aryl.

In addition to the compounds of Formula (I) described above, their pharmaceutically acceptable salts and solvates, where applicable, are also covered by this invention. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumarate, succinate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

The term “treating” or “treatment” refers to administering one or more of the secresomes to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a secresome that is required to confer the therapeutic effect. Effective doses will vary, as recognized by those skilled in the art, depending on the types of symptoms treated, route of administration, excipient usage, and the possibility of co-usage with other therapeutic treatment.

The term “subject” refers to an animal including human or non-human, such as a mammal. A human is a preferred subject.

The details of one or more embodiments of the invention are set forth in the description below. Other features, objects, and advantages of the invention will be apparent from the description and from the claims.

### DETAILED DESCRIPTION OF THE INVENTION

The compounds of this invention are prostaglandin E receptor 4 (EP4) antagonists that induce release of secresomes suitable for use as therapeutic agents in various conditions.

The compounds can be synthesized using methods well known in the art. See, for example, R. Larock, Comprehensive Organic Transformations (2^{nd} Ed., VCH Publishers 1999); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); L. Paquette, ed., Encyclopedia of Reagents for Organic Synthesis (2^{nd} ed., John Wiley and Sons 2009); N. J. O'Reilly, W. S. Derwin, H. C. Lin “Optically Pure (S)-6,7-Dimethoxy-1,2,3,4-Tetrahydro-3-Isoquinolinecarboxylic Acid and Asymmetric Hydrogenation Studies Related to Its Preparation” Synthesis 7 (1990) 550-556; J. Liu, G. Cui, M. Zhao, C. Cui, J. Ju, S. Peng “Dual-acting agents that possess reversing resistance and anticancer activities: Design, synthesis, MES-SA/Dx5 cell assay, and SAR of Benzyl 1,2,3,5,11,11a-hexahydro-3,3-dimethyl-1-oxo-6H-imidazo-[3′,4′:1,2]pyridin[3,4-b]indol-2-substituted acetates” Bioorganic & Medicinal Chemistry 15 (2007) 7773-7778; M. P. Chelopo, S. A. Pawar, M. K. Sokhela, T. Govender, H. G. Kruger, G. E. M. Maguire “Anticancer activity of ruthenium(II) arene complexes bearing 1,2,3,4-tetrahydroiso-quinoline amino alcohol ligands”, European Journal of Medicinal Chemistry 66 (2013) 407-414.

The compounds thus prepared can be initially screened using in vitro assays for their potency in inducing release of secresomes from stem cells.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

The abbreviations as used herein are provided with the corresponding definitions below.

| Abbrv | Name | Abbrv | Name |
|---|---|---|---|
| MS | mass spectrometry | NMR | nuclear magnetic resonance |
| ESI-MS | Electrospray Ionization- | HRMS | High-Resolution Mass |
| | Mass Spectrometry | | Spectrometry |
| HPLC | High-Performance | LCMS | Liquid Chromatography- |
| | Liquid Chromatography | | Mass Spectrometry |
| ACN/ | acetonitrile | ACOH | Acetic acid |
| MeCN | | | |
| tBuOH | tert-butanol | BOC | tert-butyloxycarbonyl |
| DMF | dimethylformamide | DMSO | dimethyl sulfoxide |
| DIEA/ | N,N-diisopropyl- | DCM | dichloromethane |
| DIPEA | ethylamine | | |
| EtOH | ethanol | HATU | (1-[Bis(dimethylamino)- |
| | | | methylene]-1H-1,2,3- |
| | | | triazolo[4,5-b]pyridinium |
| | | | 3-oxide |
| | | | hexafluorophosphate |
| T3P | propanephosphonic acid | | hexafluorophosphate |
| | anhydride | | benzotriazole tetramethyl |
| | | | uronium |
| TMSBr | bromotrimethylsilane | TFA | trifluoroacetic acid |

General synthetic schemes and procedures are provided below. A skilled person in the art could prepare compounds of this invention following one of these general procedures in addition to the methods known in the art.

### General Synthetic Scheme

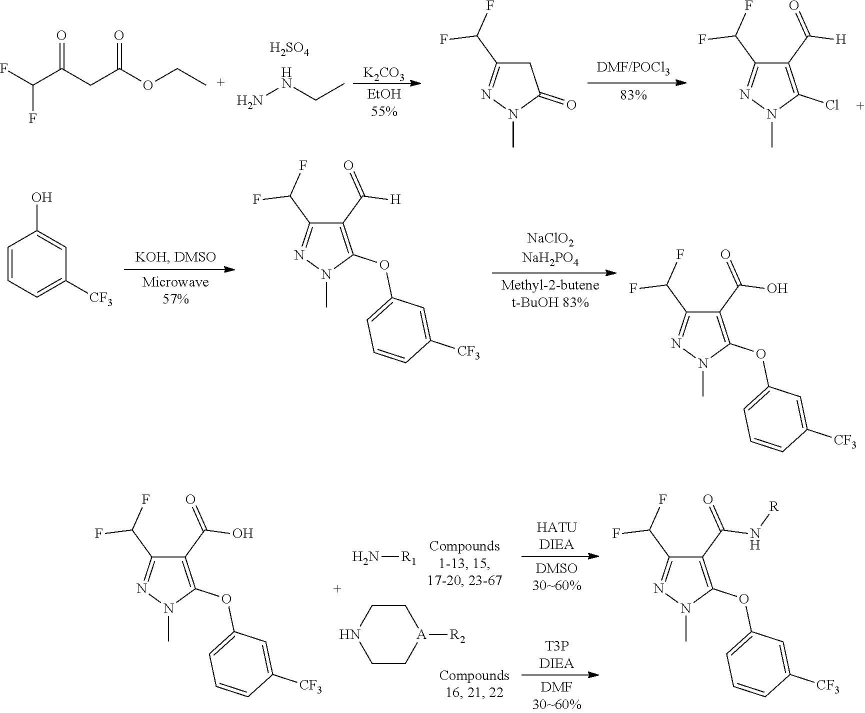

### General Procedure I

3-(Difluoromethyl)-1-methyl-5-[3-(trifluoromethyl)phenoxy]-1H-pyrazole-4-carboxylic acid (1 eq.), HATU (1.3 eq.) and DIPEA (1.5 eq.) were stirred in DMSO (1 mL) for 1 hour (h) at room temperature. Amine (2 eq.) was added to the resultant mixture and stirred for 15 h. The residue was purified suing an HPLC RP-C18 column to give a product with a yield of 19˜85%.

### General Procedure 2

3-(Difluoromethyl)-1-methyl-5-[3-(trifluoromethyl)phenoxy]-1H-pyrazole-4-carboxylic acid (1 eq.), HATU (1.2 eq.) and DIPEA (1.5 eq.) were stirred in DMSO (1 mL) for 1 h at room temperature. Amine (1.3 eq.) was added to the reaction mixture and was stirred at room temperature for 15 h. The residue was purified using an HPLC RP-C18 column to give a product with a yield of 10˜76%.

### General Procedure 3

3-(Difluoromethyl)-1-methyl-5-[3-(trifluoromethyl)phenoxy]-1H-pyrazole-4-carboxylic acid (1 eq.), HATU (1.3 eq.) and DIPEA (3 eq.) were stirred in DMSO (1 mL) for 1 h at room temperature. Amine (1.5 eq.) was added to the reaction mixture and was stirred at room temperature for 15 h. The residue was purified using an HPLC RP-C18 column to give a product with a yield of 24˜76%.

### General Procedure 4

3-(Difluoromethyl)-1-methyl-5-[3-(trifluoromethyl)phenoxy]-1H-pyrazole-4-carboxylic acid (1 eq.), T₃P (3 eq.) and DIPEA (2 eq.) were stirred in DMF (0.2 mL) for 1 h at room temperature. Amine (2 eq.) was added to the reaction mixture and was stirred at room temperature for 15 h. The residue was purified using an HPLC RP-C18 column to give a product with a yield of 18˜71%.

### Example 1: 3-(difluoromethyl)-1-methyl-N-[4-(morpholin-4-yl)phenyl]-5-[3-(trifluoromethyl)-phenoxy]-1H-pyrazole-4-carboxamide (Compound 1)

Reagents and conditions: (1) 4-(morpholin-4-yl)aniline, HATU, DIPEA, DMSO, 15 h, 68%.

3-(Difluoromethyl)-1-methyl-5-[3-(trifluoromethyl)phenoxy]-1H-pyrazole-4-carboxylic acid (70 mg, 0.2 mmol, 1 eq.), HATU (102.9 mg, 0.3 mmol, 1.3 eq.) and DIPEA (40.4 mg, 0.3 mmol, 1.5 eq.) were stirred in DMSO (1 mL) for 1 h at room temperature. 4-(Morpholin-4-yl)aniline (74.2 mg, 0.4 mmol, 2 eq.) was added to the reaction mixture and stirred at room temperature for 15 h. The residue was purified by HPLC RP-C18 column to give Compound 1 (70.5 mg, 68%).

¹H NMR (600 MHz, Acetone) δ 8.52 (br s, 1H), 7.66 (dd, J=8.4, 7.8 Hz, 1H), 7.57 (s, 1H), 7.54 (d, J=7.8 Hz, 1H), 7.38 (dd, J=8.4, 2.4 Hz, 1H), 7.36 (d, J=9.0 Hz, 2H), 7.21 (t, J=54.0 Hz, 1H), 6.85 (d, J=9.0 Hz, 1H), 3.81 (s, 3H), 3.74 (t, J=4.8 Hz, 4H), 3.05 (t, J=4.8 Hz, 4H).

ESI-MS C₂₃H₂₁F₅N₄O₃: 496.1534, found: 497.1 (M+H⁺)⁺.

HRMS (ESI, m/z): [M+H]⁺ calcd for C₂₃H₂₂F₅N₄O₃: 497.1607; found: 497.1600.

### Example 2: N-{2-[2-(2-aminoethoxy)ethoxy]ethyl}-3-(difluoromethyl)-1-methyl-5-[3-(trifluoromethyl)phenoxy]-1H-pyrazole-4-carboxamide (Compound 2)

Reagents and conditions: (1) tert-butyl {2-[2-(2-aminoethoxy)ethoxy]ethyl}carbamate, HATU, DIPEA, DMSO, 15 h, 59%. (2) TFA, DCM, 96%

### tert-Butyl [2-(2-{2-[({3-(difluoromethyl)-1-methyl-5-[3-(trifluoromethyl)phenoxy]-1H-pyrazol-4-yl}carbonyl)amino]ethoxy}ethoxy)ethyl]carbamate (Compound 2.1)

Compound 2.1 was prepared using 3-(difluoromethyl)-1-methyl-5-[3-(trifluoromethyl)-phenoxy]-1H-pyrazole-4-carboxylic acid and tert-butyl {2-[2-(2-aminoethoxy)ethoxy]ethyl}-carbamate in a manner similar to general procedure 1.

¹H NMR (600 MHz, Acetone) δ 7.70 (dd, J=8.4, 7.8 Hz, 1H), 7.59 (d, J=7.8 Hz, 1H), 7.51 (s, 1H), 7.32 (dd, J=8.4, 2.4 Hz, 1H), 7.26 (t, J=53.6 Hz, 1H), 6.89 (br s, 1H), 5.84 (br s, 1H), 3.75 (s, 3H), 3.48-3.45 (m, 3H), 3.45-3.42 (m, 2H), 3.39-3.35 (m, 3H), 3.21 (q, J=5.8 Hz, 2H), 2.81-2.80 (m, 1H), 2.78-2.76 (m, 1H), 1.38 (s, 9H).

ESI-MS C₂₄H₃₁F₅N₄O₆: 566.2164, found: 467.1 (M-BOC)⁺.

### Compound 2

To a solution of Compound 2.1 (70.1 mg, 0.1 mmol) in CH₂Cl₂ (0.5 mL) was added TFA (0.5 mL). The reaction mixture was stirred at room temperature for 2 h. After the reaction was completed, the excess of TFA was removed under vacuum. Compound 2 was obtained without further purification. (55.4 mg, 96%)

¹H NMR (600 MHz, Acetone) δ 7.70 (dd, J=8.4, 7.8 Hz, 1H), 7.58 (d, J=7.8 Hz, 1H), 7.50 (s, 1H), 7.33 (dd, J=8.4, 3.0 Hz, 1H), 7.26 (t, J=54.0 Hz, 1H), 6.91 (br s, 1H), 3.74 (s, 3H), 3.61 (t, J=6.3 Hz, 2H), 3.49-3.45 (m, 2H), 3.45-3.41 (m, 2H), 3.40-3.33 (m, 4H), 3.32 (t, J=6.3 Hz, 2H), 2.80 (br s, 2H).

ESI-MS C₁₉H₂₃F₅N₄O₄: 466.1639, found: 467.2 (M+H⁺)⁺.

HRMS (ESI, m/z): [M+H]⁺ calcd for C₁₉H₂₄F₅N₄O₄: 467.1712; found: 467.1710.

### Example 3: 3-(difluoromethyl)-1-methyl-N-[2-(pyridin-4-yl)ethyl]-5-[3-(trifluoromethyl)-phenoxy]-1H-pyrazole-4-carboxamide (Compound 3)

Reagents and conditions: 2-(pyridin-4-yl)ethanamine, HATU, DIPEA, DMSO, 15 h, 68%.

Compound 3 was prepared using 3-(difluoromethyl)-1-methyl-5-[3-(trifluoromethyl)-phenoxy]-1H-pyrazole-4-carboxylic acid and 2-(pyridin-4-yl)ethanamine in a manner similar to general procedure 2.

¹H NMR (600 MHz, Acetone) δ 8.34 (d, J=5.1 Hz, 2H), 7.67 (dd, J=8.4, 7.8 Hz, 1H), 7.58 (d, J=7.8 Hz, 1H), 7.43 (s, 1H), 7.22 (dd, J=8.4, 2.4 Hz, 1H), 7.22 (t, J=54.0 Hz, 1H), 7.05 (d, J=5.1 Hz, 2H), 3.71 (s, 3H), 3.50 (q, J=6.9 Hz, 2H), 2.70 (t, J=6.9 Hz, 2H).

ESI-MS C₂₀H₁₇F₅N₄O₂: 440.1272, found: 441.1 (M+H⁺)⁺.

HRMS (ESI, m/z): [M+H]⁺ calcd for C₂₀H₁₈F₅N₄O₂: 441.1344; found: 441.1346.

### Example 4: N-(4-carbamoylphenyl)-3-(difluoromethyl)-1-methyl-5-[3-(trifluoromethyl)-phenoxy]-1H-pyrazole-4-carboxamide (Compound 4)

Reagents and conditions: 4-aminobenzamide, HATU, DIPEA, DMSO, 15 h, 19%.

Compound 4 was prepared using 3-(difluoromethyl)-1-methyl-5-[3-(trifluoromethyl)-phenoxy]-1H-pyrazole-4-carboxylic acid and 4-aminobenzamide in a manner similar to general procedure 1.

¹H NMR (600 MHz, Acetone) δ 8.94 (br s, 1H), 7.84 (d, J=8.7 Hz, 2H), 7.65 (dd, J=8.4, 7.8 Hz, 1H), 7.60 (d, J=8.7 Hz, 2H), 7.58 (s, 1H), 7.51 (d, J=7.8 Hz, 1H), 7.41 (dd, J=8.4, 2.4 Hz, 1H), 7.18 (t, J=54.0 Hz, 1H), 3.83 (s, 3H).

ESI-MS C₂₀H₁₅F₅N₄O₃: 454.1064, found: 455.1 (M+H⁺)⁺.

HRMS (ESI, m/z): [M+H]⁺ calcd for C₂₀H₁₆F₅N₄O₃: 455.1137; found: 455.1139.

### Example 5: N-(3-chloro-5-fluorobenzyl)-3-(difluoromethyl)-1-methyl-5-[3-(trifluoromethyl)-phenoxy]-1H-pyrazole-4-carboxamide (Compound 5)

Reagents and conditions: 1-(3-chloro-5-fluorophenyl)methanamine, HATU, DIPEA, DMSO, 15 h, 41%.

Compound 5 was prepared using 3-(difluoromethyl)-1-methyl-5-[3-(trifluoromethyl)-phenoxy]-1H-pyrazole-4-carboxylic acid and 1-(3-chloro-5-fluorophenyl)methanamine in a manner similar to general procedure 2.

¹H NMR (600 MHz, Acetone) δ 7.67-7.61 (m, 2H), 7.55 (dd, J=7.8, 0.6 Hz, 1H), 7.44 (s, 1H), 7.27 (dd, J=8.4, 2.4 Hz, 1H), 7.24 (t, J=54.0 Hz, 1H), 7.03 (dt, J=8.4, 2.0 Hz, 1H), 6.97 (s, 1H), 6.80-6.77 (m, 1H), 4.42 (d, J=6.0 Hz, 2H), 3.75 (s, 3H).

ESI-MS C₂₀H₁₄ClF₆N₃O₂: 477.0679, found: 478.1 (M+H⁺)⁺.

HRMS (ESI, m/z): [M+H]⁺ calcd for C₂₀H₁₅ClF₆N₃O₂: 478.0752; found: 478.0751.

### Example 6: N-(bicyclo[1.1.1]pent-1-yl)-3-(difluoromethyl)-1-methyl-5-[3-(trifluoromethyl)phenoxy]-1H-pyrazole-4-carboxamide (Compound 6)

Reagents and conditions: Bicyclo[1.1.1]pentan-1-amine hydrochloride, HATU, DIPEA, DMSO, 15 h, 45%.

Compound 6 was prepared using 3-(difluoromethyl)-1-methyl-5-[3-(trifluoromethyl)-phenoxy]-1H-pyrazole-4-carboxylic acid and bicyclo[1.1.1]pentan-1-amine hydrochloride in a manner similar to general procedure 3.

¹H NMR (600 MHz, Acetone) δ 7.69 (dd, J=8.4, 7.8 Hz, 1H), 7.61-7.56 (m, 1H), 7.50 (s, 1H), 7.30 (dd, J=8.4, 2.4 Hz, 1H), 7.26 (br s, 1H), 7.16 (t, J=54.0 Hz, 1H), 3.76 (s, 3H), 2.30 (s, 1H), 1.87 (s, 6H).

ESI-MS C₁₈H₁₆F₅N₃O₂: 401.1163, found: 402.1 (M+H⁺)⁺.

HRMS (ESI, m/z): [M+H]⁺ calcd for C₁₈H₁₇F₅N₃O₂: 402.1235; found: 402.1234.

### Example 7: 3-(difluoromethyl)-1-methyl-N-[(1R,2S)-2-phenylcyclopropyl]-5-[3-(trifluoromethyl)phenoxy]-1H-pyrazole-4-carboxamide (Compound 7)

Reagents and conditions: (1R,2S)-2-phenylcyclopropanamine hydrochloride, HATU, DIPEA, DMSO, 15 h, 91%.

Compound 7 was prepared using 3-(difluoromethyl)-1-methyl-5-[3-(trifluoromethyl)-phenoxy]-1H-pyrazole-4-carboxylic acid and (1R,2S)-2-phenylcyclopropanamine hydrochloride in a manner similar to general procedure 3.

¹H NMR (600 MHz, Acetone) δ 7.71 (dd, J=8.4, 7.8 Hz, 1H), 7.60 (d, J=7.8 Hz, 1H), 7.52 (s, 1H), 7.32 (dd, J=8.4, 2.4 Hz, 1H), 7.24-7.20 (m, 2H), 7.19 (t, J=54.0 Hz, 1H), 7.15-7.11 (m, 1H), 7.06 (d, J=7.2 Hz, 2H), 3.78 (s, 3H), 2.81-2.77 (m, 1H), 1.66-1.61 (m, 1H), 1.10-1.06 (m, 1H), 0.92-0.87 (m, 1H).

ESI-MS C₂₂H₁₈F₅N₃O₂: 451.1319, found: 452.1 (M+H⁺)⁺.

HRMS (ESI, m/z): [M+H]⁺ calcd for C₂₂H₁₉F₅N₃O₂: 452.1392; found: 452.1397.

### Example 8: 2-({3-(difluoromethyl)-1-methyl-5-[3-(trifluoromethyl)phenoxy]-1H-pyrazol-4-yl}carbonyl)-N-methylhydrazinecarbothioamide (Compound 8)

Reagents and conditions: N-methylhydrazinecarbothioamide, 50% T₃P, DIPEA, DMF, 15 h, 24%.

Compound 8 was prepared using 3-(difluoromethyl)-1-methyl-5-[3-(trifluoromethyl)-phenoxy]-1H-pyrazole-4-carboxylic acid and N-methylhydrazinecarbothioamide in a manner similar to general procedure 4.

¹H NMR (600 MHz, Acetone) δ 8.91 (br s, 1H), 8.46 (br s, 1H), 7.71 (dd, J=8.4, 7.8 Hz, 1H), 7.60 (d, J=7.8 Hz, 1H), 7.54 (s, 1H), 7.35 (d, J=8.4 Hz, 1H), 7.21 (t, J=54.0 Hz, 1H), 7.12 (br s, 1H), 3.74 (s, 3H), 2.89 (d, J=4.2 Hz, 3H).

ESI-MS C₁₅H₁₄F₅N₅O₂S: 423.0788, found: 422.0 (M−H⁺)⁻.

HRMS (ESI, m/z): [M+H]⁺ calcd for C₁₅H₁₅F₅N₅O₂S: 424.0861; found: 424.0863.

### Example 9: 3-(difluoromethyl)-N-[2-(3,4-dihydro-2H-1,5-benzodioxepin-7-yl)ethyl]-1-methyl-5-[3-(trifluoromethyl)phenoxy]-1H-pyrazole-4-carboxamide (Compound 9)

Reagents and conditions: 2-(3,4-dihydro-2H-1,5-benzodioxepin-7-yl)ethanamine, HATU, DIPEA, DMSO, 15 h, 35%.

Compound 9 was prepared using 3-(difluoromethyl)-1-methyl-5-[3-(trifluoromethyl)-phenoxy]-1H-pyrazole-4-carboxylic acid and 2-(3,4-dihydro-2H-1,5-benzodioxepin-7-yl)ethanamine in a manner similar to general procedure 2.

¹H NMR (600 MHz, Acetone) δ 7.70 (dd, J=8.4, 7.8 Hz, 1H), 7.59 (dd, J=7.8, 1.2 Hz, 1H), 7.47 (s, 1H), 7.27 (dd, J=8.4, 2.4 Hz, 1H), 7.25 (t, J=54.0 Hz, 1H), 6.94 (br s, 1H), 6.76 (d, J=8.1 Hz, 1H), 6.72 (d, J=2.1 Hz, 1H), 6.64 (dd, J=8.1, 2.1 Hz, 1H), 4.16-4.07 (m, 4H), 3.73 (s, 3H), 3.45-3.38 (m, 2H), 2.56 (t, J=7.5 Hz, 2H), 2.14 (quin, J=5.6 Hz, 2H).

ESI-MS C₂₄H₂₂F₅N₃O₄: 511.1530, found: 512.2 (M+H⁺)⁺.

HRMS (ESI, m/z): [M+H]⁺ calcd for C₂₄H₂₃F₅N₃O₄: 512.1603; found: 512.1594.

### Example 10: 3-(difluoromethyl)-1-methyl-5-[3-(trifluoromethyl)phenoxy]-N-[2-(3,4,5-trihydroxyphenyl)ethyl]-1H-pyrazole-4-carboxamide (Compound 10)

Reagents and conditions: 5-(2-aminoethyl)benzene-1,2,3-triol hydrochloride, HATU, DIPEA, DMSO, 15 h, 40%.

Compound 10 was prepared using 3-(difluoromethyl)-1-methyl-5-[3-(trifluoromethyl)-phenoxy]-1H-pyrazole-4-carboxylic acid and 5-(2-aminoethyl)benzene-1,2,3-triol hydrochloride in a manner similar to general procedure 3.

¹H NMR (600 MHz, Acetone) δ 7.70-7.63 (m, 2H), 7.55 (d, J=7.8 Hz, 1H), 7.42 (s, 1H), 7.25 (t, J=54.0 Hz, 1H), 7.23 (dd, J=8.4, 2.4 Hz, 1H), 6.86 (br s, 1H), 6.17 (s, 2H), 3.72 (s, 3H), 3.38-3.29 (m, 2H), 2.41 (t, J=7.5 Hz, 2H).

ESI-MS C₂₁H₁₈F₅N₃O₅: 487.1167, found: 488.1 (M+H⁺)⁺.

HRMS (ESI, m/z): [M+H]⁺ calcd for C₂₁H₁₉F₅N₃O₅: 488.1239; found: 488.1237.

### Example 11: N-(2-{2-[2-(2-azidoethoxy)ethoxy]ethoxy}ethyl)-3-(difluoromethyl)-1-methyl-5-[3-(trifluoromethyl)phenoxy]-1H-pyrazole-4-carboxamide (Compound 11)

Reagents and conditions: 2-{2-[2-(2-azidoethoxy)ethoxy]ethoxy}ethanamine, HATU, DIPEA, DMSO, 15 h, 50%.

Compound 11 was prepared using 3-(difluoromethyl)-1-methyl-5-[3-(trifluoromethyl)-phenoxy]-1H-pyrazole-4-carboxylic acid and 2-{2-[2-(2-azidoethoxy)ethoxy]ethoxy}ethanamine in a manner similar to general procedure 2.

¹H NMR (600 MHz, Acetone) δ 7.70 (dd, J=8.4, 7.8 Hz, 1H), 7.61-7.56 (m, 1H), 7.50 (s, 1H), 7.33 (dd, J=8.4, 3.0 Hz, 1H), 7.26 (t, J=54.0 Hz, 1H), 6.88 (br s, 1H), 3.75 (s, 3H), 3.69-3.64 (m, 2H), 3.64-3.56 (m, 4H), 3.51-3.43 (m, 4H), 3.41-3.32 (m, 6H).

ESI-MS C₂₁H₂₅F₅N₆O₅: 536.1807, found: 537.2 (M+H⁺)⁺.

HRMS (ESI, m/z): [M+Na]⁺ calcd for C₂₁H₂₅F₅N₆O₅Na: 559.1699; found: 559.1698.

### Example 12: 3-(difluoromethyl)-1-methyl-N-(2-oxo-2,3-dihydro-1H-indol-5-yl)-5-[3-(trifluoromethyl)phenoxy]-1H-pyrazole-4-carboxamide (Compound 12)

Reagents and conditions: 5-amino-1,3-dihydro-2H-indol-2-one, 50% T₃P, DIPEA, DMF, 15 h, 58%.

Compound 12 was prepared using 3-(difluoromethyl)-1-methyl-5-[3-(trifluoromethyl)-phenoxy]-1H-pyrazole-4-carboxylic acid and 5-amino-1,3-dihydro-2H-indol-2-one in a manner similar to general procedure 4.

¹H NMR (600 MHz, Acetone) δ 9.26 (br s, 1H), 8.63 (br s, 1H), 7.66 (dd, J=8.4, 7.8 Hz, 1H), 7.57 (s, 1H), 7.56-7.52 (m, 1H), 7.43 (s, 1H), 7.39 (dd, J=8.4, 2.4 Hz, 1H), 7.23 (dd, J=8.4, 2.4 Hz, 1H), 7.19 (t, J=54.0 Hz, 1H), 6.75 (d, J=8.4 Hz, 1H), 3.81 (s, 3H), 3.39 (s, 2H).

ESI-MS C₂₁H₁₅F₅N₄O₃: 466.1064, found: 465.0 (M−H⁺)⁻.

HRMS (ESI, m/z): [M+H]⁺ calcd for C₂₁H₁₆F₅N₄O₃: 467.1137; found: 467.1145.

### Example 13: 3-(difluoromethyl)-N-[2-(2-hydroxyethoxy)ethyl]-1-methyl-5-[3-(trifluoromethyl)-phenoxy]-1H-pyrazole-4-carboxamide (Compound 13)

Reagents and conditions: 2-(2-aminoethoxy)ethanol, HATU, DIPEA, DMSO, 15 h, 67%.

Compound 13 was prepared using 3-(difluoromethyl)-1-methyl-5-[3-(trifluoromethyl)-phenoxy]-1H-pyrazole-4-carboxylic acid and 2-(2-aminoethoxy)ethanol in a manner similar to general procedure 2.

¹H NMR (600 MHz, Acetone) δ 7.69 (dd, J=8.4, 7.8 Hz, 1H), 7.60-7.54 (m, 1H), 7.50 (s, 1H), 7.31 (dd, J=8.4, 2.4 Hz, 1H), 7.24 (t, J=54.0 Hz, 1H), 6.95 (br s, 1H), 3.74 (s, 3H), 3.60 (br s, 1H), 3.56-3.49 (m, 2H), 3.42-3.32 (m, 6H).

ESI-MS C₁₇H₁₈F₅N₃O₄: 423.1217, found: 424.1 (M+H⁺)⁺.

HRMS (ESI, m/z): [M+Na]⁺ calcd for C₁₇H₁₈F₅N₃O₄Na: 446.1109; found: 446.1115.

### Example 14: [3-(methoxycarbonyl)bicyclo[1.1.1]pent-1-yl]methyl 3-(difluoromethyl)-1-methyl-5-[3-(trifluoromethyl)phenoxy]-1H-pyrazole-4-carboxylate (Compound 14)

Reagents and conditions: Methyl 3-(hydroxymethyl)bicyclo[1.1.1]pentane-1-carboxylate, EDCl, DMAP, DMSO, 15 h, 14%.

Compound 14 were prepared by mixing 3-(difluoromethyl)-1-methyl-5-[3-(trifluoromethyl)-phenoxy]-1H-pyrazole-4-carboxylic acid (35 mg, 0.1 mmol, 1 eq.), EDCl (25.9 mg, 0.1 mmol, 1.3 eq.), DMAP (6 mg, 0.1 mmol, 0.5 eq.) and methyl 3-(hydroxymethyl)bicyclo[1.1.1]pentane-1-carb-oxylate (17.9 mg, 0.1 mmol, 1.1 eq.) were stirred in DMSO (1.0 mL) at room temperature for 15 h. The residue was purified by HPLC RP-C18 column to give Compound 14 (7 mg, 14%).

¹H NMR (400 MHz, cdcl₃) δ 7.49 (t, J=8.0 Hz, 1H), 7.43 (d, J=8.0 Hz, 1H), 7.23 (s, 1H), 7.08-7.00 (m, 1H), 7.04 (t, J=54.0 Hz, 1H), 4.10 (s, 2H), 3.77 (s, 3H), 3.66 (s, 3H), 1.84 (s, 6H).

ESI-MS C₂₁H₁₉F₅N₂O₅: 474.1214, found: 475.1 (M+H⁺)⁺.

HRMS (ESI, m/z): [M+H]⁺ calcd for C₂₁H₂₀F₅N₂O₅: 475.1287; found: 475.1281.

### Example 15: 3-(difluoromethyl)-1-methyl-N-[2-(4-sulfamoylphenyl)ethyl]-5-[3-(trifluoro-methyl)phenoxy]-1H-pyrazole-4-carboxamide (Compound 15)

Reagents and conditions: 4-(2-aminoethyl)benzenesulfonamide, HATU, DIPEA, DMSO, 15 h, 72%.

Compound 15 was prepared using 3-(difluoromethyl)-1-methyl-5-[3-(trifluoromethyl)-phenoxy]-1H-pyrazole-4-carboxylic acid and 4-(2-aminoethyl)benzenesulfonamide in a manner similar to general procedure 2.

¹H NMR (600 MHz, Acetone) δ 7.73 (d, J=8.4 Hz, 2H), 7.67 (dd, J=8.4, 7.8 Hz, 1H), 7.58 (dd, J=7.8, 0.6 Hz, 1H), 7.45 (s, 1H), 7.26 (d, J=8.4 Hz, 2H), 7.23 (dd, J=8.4, 2.4 Hz, 1H), 7.23 (t, J=54.0 Hz, 1H), 7.06 (br s, 1H), 6.49 (br s, 2H), 3.72 (s, 3H), 3.51-3.46 (m, 2H), 2.75 (t, J=7.5 Hz, 2H).

ESI-MS C₂₁H₁₉F₅N₄O₄S: 518.1047, found: 519.1 (M+H⁺)⁺.

HRMS (ESI, m/z): [M+H]⁺ calcd for C₂ᵢH₂₀F₅N₄O₄S: 519.1120; found: 519.1120.

### Example 16: {3-(difluoromethyl)-1-methyl-5-[3-(trifluoromethyl)phenoxy]-1H-pyrazol-4-yl}[4-(oxetan-3-yl)piperazin-1-yl]methanone (Compound 16)

Reagents and conditions: 1-(oxetan-3-yl)piperazine, 50% T₃P, DIPEA, DMF, 15 h, 71%.

Compound 16 was prepared using 3-(difluoromethyl)-1-methyl-5-[3-(trifluoromethyl)-phenoxy]-1H-pyrazole-4-carboxylic acid and 1-(oxetan-3-yl)piperazine in a manner similar to general procedure 4.

¹H NMR (600 MHz, Acetone) δ 7.67 (dd, J=8.4, 7.8 Hz, 1H), 7.57 (d, J=7.8 Hz, 1H), 7.49 (s, 1H), 7.39 (dd, J=8.4, 2.4 Hz, 1H), 6.81 (t, J=54.0 Hz, 1H), 4.52 (t, J=6.3 Hz, 2H), 4.44 (t, J=6.3 Hz, 2H), 3.80 (s, 3H), 3.41-3.32 (m, 5H), 2.12 (br s, 4H).

ESI-MS C₂₀H₂₁F₅N₄O₃: 460.1534, found: 461.2 (M+H⁺)⁺.

HRMS (ESI, m/z): [M+H]⁺ calcd for C₂₀H₂₂F₅N₄O₃: 461.1607; found: 461.1611.

### Example 17: Diethyl{2-[({3-(difluoromethyl)-1-methyl-5-[3-(trifluoromethyl)phenoxy]-1H-pyrazol-4-yl}carbonyl)amino]ethyl}phosphonate (Compound 17)

Reagents and conditions: Diethyl (2-aminoethyl)phosphonate hydrochloride, HATU, DIPEA, DMSO, 15 h, 39%.

Compound 17 was prepared using 3-(difluoromethyl)-1-methyl-5-[3-(trifluoromethyl)-phenoxy]-1H-pyrazole-4-carboxylic acid and diethyl (2-aminoethyl)phosphonate hydrochloride in a manner similar to general procedure 3.

¹H NMR (600 MHz, Acetone) δ 7.68 (dd, J=8.4, 7.8 Hz, 1H), 7.57 (d, J=7.8 Hz, 1H), 7.53 (s, 1H), 7.37 (br s, 1H), 7.29 (d, J=8.4 Hz, 1H), 7.27 (t, J=54.0 Hz, 1H), 4.07-3.94 (m, 4H), 3.74 (s, 3H), 3.48-3.40 (m, 2H), 1.80 (dt, J=17.4, 7.2 Hz, 2H), 1.24 (t, J=6.9 Hz, 6H).

ESI-MS C₁₉H₂₃F₅N₃O₅P: 499.1295, found: 500.1 (M+H⁺)⁺.

HRMS (ESI, m/z): [M+H]⁺ calcd for C₁₉H₂₄F₅N₃O₅P: 500.1368; found: 500.1368.

### Example 18: {2-[({3-(difluoromethyl)-1-methyl-5-[3-(trifluoromethyl)phenoxy]-1H-pyrazol-4-yl}carbonyl)amino]ethyl}phosphonic acid (Compound 18)

Reagents and conditions: Diethyl{2-[({3-(difluoromethyl)-1-methyl-5-[3-(trifluoromethyl)-phenoxy]-1H-pyrazol-4-yl}carbonyl)amino]ethyl}phosphonate, TMSBr, DCM, 15 h, 80%.

To a solution of Compound 17 (744.7 mg, 1.5 mmol, 1 eq.) in CH₂Cl₂ (2.8 mL) was added TMSBR (2.8 mL, 20.9 mmol, 14 eq.). The reaction mixture was stirred at room temperature for overnight. After the reaction was completed, the solvent was removed under vacuum. The residue was purified by HPLC RP-C18 column to give Compound 18 (531.2 mg, 80%).

¹H NMR (600 MHz, Acetone) δ 7.67 (dd, J=8.4, 7.8 Hz, 1H), 7.55 (d, J=7.8 Hz, 1H), 7.46 (s, 1H), 7.31 (d, J=8.4 Hz, 1H), 7.25 (t, J=54.0 Hz, 1H), 3.72 (s, 3H), 3.48 (br s, 2H), 1.81 (br s, 2H).

ESI-MS C₁₅H₁₅F₅N₃O₅P: 443.0669, found: 442.0 (M−H⁺)⁻.

HRMS (ESI, m/z): [M+H]⁺ calcd for C₁₅H₁₆F₅N₃O₅P: 444.0742; found: 444.0743.

### Example 19: 2-({3-(difluoromethyl)-1-methyl-5-[3-(trifluoromethyl)phenoxy]-1H-pyrazol-4-yl}carbonyl)hydrazinecarboxamide (Compound 19)

Reagents and conditions: Hydrazinecarboxamide hydrochloride, HATU, DIPEA, DMSO, 15 h, 62%.

Compound 19 was prepared using 3-(difluoromethyl)-1-methyl-5-[3-(trifluoromethyl)-phenoxy]-1H-pyrazole-4-carboxylic acid and hydrazinecarboxamide hydrochloride in a manner similar to general procedure 3.

¹H NMR (600 MHz, Acetone) δ 7.68 (dd, J=8.4, 7.8 Hz, 1H), 7.57 (d, J=7.8 Hz, 1H), 7.52 (s, 1H), 7.33 (dd, J=8.4, 2.4 Hz, 1H), 7.28 (br s, 1H), 7.22 (t, J=54.0 Hz, 1H), 5.42 (br s, 2H), 3.75 (s, 3H).

ESI-MS C₁₄H₁₂F₅N₅O₃: 393.0860, found: 392.0 (M−H⁺)⁻.

HRMS (ESI, m/z): [M−H]⁻ calcd for C₁₄H₁₁F₅N₅O₃: 392.0788; found: 392.0789.

### Example 20: 3-(difluoromethyl)-N-[1,3-dihydroxy-2-(hydroxymethyl)propan-2-yl]-1-methyl-5-[3-(trifluoromethyl)phenoxy]-1H-pyrazole-4-carboxamide (Compound 20)

Reagents and conditions: 2-amino-2-(hydroxymethyl)propane-1,3-diol, HATU, DIPEA, DMSO, 15 h, 47%.

Compound 20 was prepared using 3-(difluoromethyl)-1-methyl-5-[3-(trifluoromethyl)-phenoxy]-1H-pyrazole-4-carboxylic acid and 2-amino-2-(hydroxymethyl)propane-1,3-diol in a manner similar to general procedure 2.

¹H NMR (600 MHz, Acetone) δ 7.68 (dd, J=8.4, 7.8 Hz, 1H), 7.58 (d, J=7.8 Hz, 1H), 7.47 (s, 1H), 7.31 (dd, J=8.4, 2.4 Hz, 1H), 7.25 (t, J=54.0 Hz, 1H), 7.23 (br s, 1H), 4.33 (br s, 3H), 3.77 (s, 3H), 3.53 (s, 6H).

ESI-MS C₁₇H₁₈F₅N₃O₅: 439.1167, found: 438.0 (M−H⁺)⁻.

HRMS (ESI, m/z): [M+H]⁺ calcd for C₁₇H₁₉F₅N₃O₅: 440.1239; found: 440.1240.

### Example 21: 4-({3-(difluoromethyl)-1-methyl-5-[3-(trifluoromethyl)phenoxy]-1H-pyrazol-4-yl}carbonyl)piperazine-1-carboximidamide (Compound 21)

Reagents and conditions: 2-(piperazin-1-yl)-1,3,5-triazine, HATU, DIPEA, DMSO, 15 h, 29%.

Compound 21 was prepared using 3-(difluoromethyl)-1-methyl-5-[3-(trifluoromethyl)-phenoxy]-1H-pyrazole-4-carboxylic acid and 2-(piperazin-1-yl)-1,3,5-triazine in a manner similar to general procedure 1.

¹H NMR (600 MHz, Acetone) δ 7.67 (dd, J=8.4, 7.8 Hz, 1H), 7.56 (d, J=7.8 Hz, 1H), 7.50 (s, 1H), 7.40 (dd, J=8.4, 2.4 Hz, 1H), 6.83 (t, J=54.0 Hz, 1H), 3.81 (s, 3H), 3.40 (br s, 4H), 3.23 (br s, 4H).

ESI-MS C₁₈H₁₉F₅N₆O₂: 446.1490, found: 447.1 (M+H⁺)⁺.

HRMS (ESI, m/z): [M+H]⁺ calcd for C₁₈H₂₀F₅N₆O₂: 447.1562; found: 447.1563.

### Example 22: {3-(difluoromethyl)-1-methyl-5-[3-(trifluoromethyl)phenoxy]-1H-pyrazol-4-yl}(1,1-dioxidothiomorpholin-4-yl)methanone (Compound 22)

Reagents and conditions: Thiomorpholine 1,1-dioxide, HATU, DIPEA, DMSO, 15 h, 42%.

Compound 22 was prepared using 3-(difluoromethyl)-1-methyl-5-[3-(trifluoromethyl)-phenoxy]-1H-pyrazole-4-carboxylic acid and thiomorpholine 1,1-dioxide in a manner similar to general procedure 2.

¹H NMR (600 MHz, Acetone) δ 7.69 (dd, J=8.4, 7.8 Hz, 1H), 7.59 (d, J=7.8 Hz, 1H), 7.54 (s, 1H), 7.44 (dd, J=8.4, 3.0 Hz, 1H), 6.87 (t, J=54.0 Hz, 1H), 3.84 (br s, 4H), 3.82 (s, 3H), 2.95 (t, J=5.4 Hz, 4H).

ESI-MS C₁₇H₁₆F₅N₃O₄S: 453.0782, found: 454.1 (M+H⁺)⁺.

HRMS (ESI, m/z): [M+H]⁺ calcd for C₁₇H₁₇F₅N₃O₄S: 454.0854; found: 454.0856.

### Example 23: 3-(difluoromethyl)-N-(4-methoxyphenyl)-1-methyl-5-[3-(trifluoromethyl)-phenoxy]-1H-pyrazole-4-carbohydrazide (Compound 23)

Reagents and conditions: (4-methoxyphenyl)hydrazine hydrochloride, HATU, DIPEA, DMSO, 15 h, 60%.

Compound 23 was prepared using 3-(difluoromethyl)-1-methyl-5-[3-(trifluoromethyl)-phenoxy]-1H-pyrazole-4-carboxylic acid and (4-methoxyphenyl)hydrazine hydrochloride in a manner similar to general procedure 3.

¹H NMR (600 MHz, Acetone) δ 8.81 (br d, J=3.9 Hz, 1H), 7.72 (dd, J=8.4, 7.8 Hz, 1H), 7.64 (d, J=7.8 Hz, 1H), 7.55 (s, 1H), 7.33 (dd, J=8.4, 2.4 Hz, 1H), 7.18 (t, J=54.0 Hz, 1H), 6.78 (br d, J=3.9 Hz, 1H), 6.62-6.55 (m, 2H), 6.49-6.43 (m, 2H), 3.80 (s, 3H), 3.65 (s, 3H).

ESI-MS C₂₀H₁₇F₅N₄O₃: 456.1221, found: 455.0 (M−H⁺)⁻.

HRMS (ESI, m/z): [M+H]⁺ calcd for C₂₀H₁₈F₅N₄O₃: 457.1294; found: 457.1292.

### Example 24: 3-(difluoromethyl)-N-[3-(hydroxymethyl)benzyl]-1-methyl-5-[3-(trifluoromethyl)phenoxy]-1H-pyrazole-4-carboxamide (Compound 24)

Reagents and conditions: [3-(aminomethyl)phenyl]methanol, HATU, DIPEA, DMSO, 15 h, 76%.

Compound 24 was prepared using 3-(difluoromethyl)-1-methyl-5-[3-(trifluoromethyl)-phenoxy]-1H-pyrazole-4-carboxylic acid and [3-(aminomethyl)phenyl]methanol in a manner similar to general procedure 2.

¹H NMR (600 MHz, Acetone) δ 7.65 (dd, J=8.4, 7.8 Hz, 1H), 7.56 (d, J=7.8 Hz, 1H), 7.44 (s, 1H), 7.41 (br s, 1H), 7.27 (t, J=54.0 Hz, 1H), 7.25 (dd, J=8.4, 2.4 Hz, 1H), 7.17 (d, J=7.2 Hz, 1H), 7.13-7.08 (m, 2H), 6.89 (d, J=7.2 Hz, 1H), 4.51 (s, 2H), 4.39 (d, J=6.0 Hz, 2H), 3.74 (s, 3H).

ESI-MS C₂₁H₁₈F₅N₃O₃: 455.1268, found: 456.1 (M+H⁺)⁺.

HRMS (ESI, m/z): [M+H]⁺ calcd for C₂₁H₁₉F₅N₃O₃: 456.1341; found: 456.1431.

### Example 25: N²,N²-bis(carboxymethyl)-N⁶-({3-(difluoromethyl)-1-methyl-5-[3-(trifluoromethyl)phenoxy]-1H-pyrazol-4-yl}carbonyl)-D-lysine (Compound 25)

Reagents and conditions: N²,N²-bis(carboxymethyl)-D-lysine, HATU, DIPEA, DMSO, 15 h, 10%.

Compound 25 was prepared by mixing 3-(difluoromethyl)-1-methyl-5-[3-trifluoromethyl)-phenoxy]-1H-pyrazole-4-carboxylic acid (70 mg, 0.2 mmol, 1 eq.), HATU (79.2 mg, 0.2 mmol, 1 eq.) and DIPEA (107.6 mg, 0.8 mmol, 4 eq.) in DMSO (1.4 mL) for 1 h at room temperature. N²,N²-bis(carboxymethyl)-D-lysine (81.9 mg, 0.3 mmol, 1.5 eq.) was added to the reaction mixture and was stirred at room temperature for 15 h. The residue was purified by HPLC RP-C18 column to give Compound 25 (12.5 mg, 10%).

¹H NMR (600 MHz, Acetone) δ 7.68 (dd, J=8.4, 7.8 Hz, 1H), 7.57 (dd, J=7.8, 0.6 Hz, 1H), 7.48 (s, 1H), 7.29 (dd, J=8.4, 2.4 Hz, 1H), 7.24 (t, J=54.3 Hz, 1H), 6.95 (br s, 1H), 3.74 (s, 3H), 3.63 (q, J=18.6 Hz, 4H), 3.37 (dd, J=8.7, 6.3 Hz, 1H), 3.19 (t, J=6.0 Hz, 2H), 1.76-1.67 (m, 1H), 1.63-1.54 (m, 1H), 1.41-1.29 (m, 4H).

ESI-MS C₂₃H₂₅F₅N₄O₈: 580.1539, found: 579.0 (M−H⁺)⁻.

HRMS (ESI, m/z): [M+H]⁺ calcd for C₂₃H₂₆F₅N₄O₈: 581.1665; found: 581.1674.

### Example 26: 3-(difluoromethyl)-1-methyl-N-(pyridin-4-ylmethyl)-5-[3-(trifluoromethyl)-phenoxy]-1H-pyrazole-4-carboxamide (Compound 26)

Reagents and conditions: 1-(pyridin-4-yl)methanamine, HATU, DIPEA, DMSO, 15 h, 81%.

Compound 26 was prepared using 3-(difluoromethyl)-1-methyl-5-[3-(trifluoromethyl)-phenoxy]-1H-pyrazole-4-carboxylic acid and 1-(pyridin-4-yl)methanamine in a manner similar to general procedure 1.

¹H NMR (600 MHz, Acetone) δ 8.32 (d, J=6.0 Hz, 2H), 7.68 (dd, J=8.4, 7.8 Hz, 1H), 7.63 (br s, 1H), 7.59 (dd, J=7.8, 1.2 Hz, 1H), 7.47 (s, 1H), 7.29 (dd, J=8.4, 3.0 Hz, 1H), 7.24 (t, J=54.0 Hz, 1H), 6.96 (d, J=6.0 Hz, 2H), 4.43 (d, J=6.0 Hz, 2H), 3.77 (s, 3H).

ESI-MS C₁₉H₁₅F₅N₄O₂: 426.1115, found: 427.1 (M+H⁺)⁺.

HRMS (ESI, m/z): [M+H]⁺ calcd for C₁₉H₁₆F₅N₄O₂: 427.1188; found: 427.1194.

### Example 27: 3-(difluoromethyl)-N-[3-methoxy-5-(trifluoromethyl)phenyl]-1-methyl-5-[3-(trifluoromethyl)phenoxy]-1H-pyrazole-4-carboxamide (Compound 27)

Reagents and conditions: (1) 3-methoxy-5-(trifluoromethyl)aniline, 50% T₃P, DIPEA, DMF, 15 h, 18%.

Compound 27 was prepared using 3-(difluoromethyl)-1-methyl-5-[3-(trifluoromethyl)-phenoxy]-1H-pyrazole-4-carboxylic acid and 3-methoxy-5-(trifluoromethyl)aniline in a manner similar to general procedure 4.

¹H NMR (600 MHz, Acetone) δ 9.04 (br s, 1H), 7.64 (dd, J=8.4, 7.8 Hz, 1H), 7.59 (s, 1H), 7.52-7.50 (m, 1H), 7.48 (s, 1H), 7.43-7.39 (m, 2H), 7.16 (t, J=54.0 Hz, 1H), 6.90 (s, 1H), 3.83 (s, 3H), 3.83 (s, 3H).

ESI-MS C₂₁H₁₅F₈N₃O₃: 509.0986, found: 508.0 (M−H⁺)⁻.

HRMS (ESI, m/z): [M+H]⁺ calcd for C₂₁H₁₆F₈N₃O₃: 510.1058; found: 510.1062.

### Example 28: 3-(difluoromethyl)-1-methyl-N-(thiophen-2-ylcarbonyl)-5-[3-(trifluoromethyl)phenoxy]-1H-pyrazole-4-carbohydrazide (Compound 28)

Reagents and conditions: Thiophene-2-carbohydrazide, HATU, DIPEA, DMSO, 15 h, 73%.

Compound 28 was prepared using 3-(difluoromethyl)-1-methyl-5-[3-(trifluoromethyl)-phenoxy]-1H-pyrazole-4-carboxylic acid and thiophene-2-carbohydrazide in a manner similar to general procedure 1.

¹H NMR (600 MHz, Acetone) δ 7.80 (d, J=3.8 Hz, 1H), 7.75 (d, J=3.8 Hz, 1H), 7.69 (dd, J=8.4, 7.8 Hz, 1H), 7.58 (d, J=7.8 Hz, 1H), 7.52 (s, 1H), 7.33 (dd, J=8.4, 2.4 Hz, 1H), 7.23 (t, J=54.0 Hz, 1H), 7.17-7.14 (m, 1H), 3.75 (s, 3H).

ESI-MS C₁₈H₁₃F₅N₄O₃S: 460.0629, found: 459.0 (M−H⁺)⁻.

HRMS (ESI, m/z): [M−H]⁻ calcd for C₁₈H₁₂F₅N₄O₃S: 459.0556; found: 459.0604.

### Example 29: 2-({3-(difluoromethyl)-1-methyl-5-[3-(trifluoromethyl)phenoxy]-1H-pyrazol-4-yl}carbonyl)hydrazinecarbothioamide (Compound 29)

Reagents and conditions: Hydrazinecarbothioamide, 50% T₃P, DIPEA, DMF, 15 h, 46%.

Compound 29 was prepared using 3-(difluoromethyl)-1-methyl-5-[3-(trifluoromethyl)-phenoxy]-1H-pyrazole-4-carboxylic acid and hydrazinecarbothioamide in a manner similar to general procedure 4.

¹H NMR (400 MHz, cdcl₃) δ 7.53 (dd, J=8.4, 7.6 Hz, 1H), 7.48 (d, J=7.6 Hz, 1H), 7.25 (s, 1H), 7.14 (d, J=8.4 Hz, 1H), 6.89 (t, J=54.0 Hz, 1H), 6.25 (br s, 2H), 3.72 (s, 3H).

ESI-LRMS C₁₄H₁₂F₅N₅O₂S: 409.0632, found: 408.1 (M−H⁺)⁻.

HRMS (ESI, m/z): [M+H]⁺ calcd for C₁₄H₁₃F₅N₅O₂S: 410.0705; found: 410.0704.

### Example 30: 3-(difluoromethyl)-N-(furan-2-ylcarbonyl)-1-methyl-5-[3-(trifluoromethyl)-phenoxy]-1H-pyrazole-4-carbohydrazide (Compound 30)

Reagents and conditions: Furan-2-carbohydrazide, 50% T₃P, DIPEA, DMF, 15 h, 43%.

Compound 30 was prepared using 3-(difluoromethyl)-1-methyl-5-[3-(trifluoromethyl)-phenoxy]-1H-pyrazole-4-carboxylic acid and furan-2-carbohydrazide in a manner similar to general procedure 4.

¹H NMR (400 MHz, Acetone) δ 9.47 (br s, 1H), 8.88 (br s, 1H), 7.73-7.65 (m, 2H), 7.58 (d, J=7.2 Hz, 1H), 7.52 (s, 1H), 7.33 (dd, J=8.4, 2.0 Hz, 1H), 7.23 (t, J=54.0 Hz, 1H), 7.14 (d, J=3.6 Hz, 1H), 6.60 (dd, J=3.6, 2.0 Hz, 1H), 3.75 (s, 3H).

ESI-MS C₁₈H₁₃F₅N₄O₄: 444.0857, found: 443.0 (M−H⁺)⁻.

HRMS (ESI, m/z): [M+H]⁺ calcd for C₁₈H₁₄F₅N₄O₄: 445.0930; found: 445.0935.

### Example 31: N′-({3-(difluoromethyl)-1-methyl-5-[3-(trifluoromethyl)phenoxy]-1H-pyrazol-4-yl}carbonyl)pyridine-4-carbohydrazide (Compound 31)

Reagents and conditions: Pyridine-4-carbohydrazide, 50% T₃P, DIPEA, DMF, 15 h, 47%.

Compound 31 was prepared using 3-(difluoromethyl)-1-methyl-5-[3-(trifluoromethyl)-phenoxy]-1H-pyrazole-4-carboxylic acid and pyridine-4-carbohydrazide in a manner similar to general procedure 4.

¹H NMR (600 MHz, Acetone) δ 8.73 (dd, J=4.2, 1.8 Hz, 2H), 7.75 (dd, J=4.2, 1.8 Hz, 2H), 7.70 (dd, J=8.4, 7.8 Hz, 1H), 7.59 (d, J=7.8 Hz, 1H), 7.53 (s, 1H), 7.35 (dd, J=8.4, 2.4 Hz, 1H), 7.22 (t, J=54.0 Hz, 1H), 3.76 (s, 3H).

ESI-MS C₁₉H₁₄F₅N₅O₃: 455.1017, found: 456.1 (M+H⁺)⁻.

HRMS (ESI, m/z): [M+H]⁺ calcd for C₁₉H₁₅F₅N₅O₃: 456.1090; found: 456.1093.

### Example 32: 3-(difluoromethyl)-1-methyl-5-[3-(trifluoromethyl)phenoxy]-N-(2,4,6-trimethylphenyl)-1H-pyrazole-4-carbohydrazide (Compound 32)

Reagents and conditions: (2,4,6-trimethylphenyl)hydrazine hydrochloride, HATU, DIPEA, DMSO, 15 h, 76%.

Compound 32 was prepared using 3-(difluoromethyl)-1-methyl-5-[3-(trifluoromethyl)-phenoxy]-1H-pyrazole-4-carboxylic acid and (2,4,6-trimethylphenyl)hydrazine hydrochloride in a manner similar to general procedure 3.

¹H NMR (600 MHz, Acetone) δ 8.53 (br s, 1H), 7.63 (dd, J=8.4, 7.8 Hz, 1H), 7.55 (d, J=7.8 Hz, 1H), 7.47 (s, 1H), 7.22 (dd, J=8.4, 2.4 Hz, 1H), 7.18 (t, J=54.0 Hz, 1H), 6.64 (s, 2H), 6.31 (br t, J=3.9 Hz, 1H), 3.72 (s, 3H), 2.15 (s, 6H), 2.12 (s, 3H).

ESI-MS C₂₂H₂₁F₅N₄O₂: 468.1585, found: 469.1 (M+H⁺)⁺.

HRMS (ESI, m/z): [M+H]⁺ calcd for C₂₂H₂₂F₅N₄O₂: 469.1657; found: 469.1666.

### Example 33: N-(1,3-benzothiazol-2-yl)-3-(difluoromethyl)-1-methyl-5-[3-(trifluoromethyl)phenoxy]-1H-pyrazole-4-carbohydrazide (Compound 33)

Reagents and conditions: 2-hydrazinyl-1,3-benzothiazole, HATU, DIPEA, DMSO, 15 h, 58%.

Compound 33 was prepared using 3-(difluoromethyl)-1-methyl-5-[3-(trifluoromethyl)-phenoxy]-1H-pyrazole-4-carboxylic acid and 2-hydrazinyl-1,3-benzothiazole in a manner similar to general procedure 2.

¹H NMR (600 MHz, Acetone) δ 7.91-7.88 (m, 1H), 7.78-7.74 (m, 1H), 7.57 (dd, J=8.4, 7.8 Hz, 1H), 7.51 (s, 1H), 7.47-7.43 (m, 1H), 7.42-7.37 (m, 2H), 7.35-7.31 (m, 1H), 7.07 (t, J=54.6 Hz, 1H), 3.84 (s, 3H).

ESI-MS C₂₀H₁₄F₅N₅O₂S: 483.0788, found: 484.1 (M+H⁺)⁺.

HRMS (ESI, m/z): [M+H]⁺ calcd for C₂₀H₁₅F₅N₅O₂S: 484.0861; found: 484.0861.

### Example 34: N-(1,3-benzothiazol-2-yl)-3-(difluoromethyl)-1-methyl-5-[3-(trifluoromethyl)phenoxy]-1H-pyrazole-4-carbohydrazide (Compound 34)

Reagents and conditions: 2-hydrazinyl-1,3-benzothiazole, HATU, DIPEA, DMSO, 15 h, 10%.

Compound 34 was prepared using 3-(difluoromethyl)-1-methyl-5-[3-(trifluoromethyl)-phenoxy]-1H-pyrazole-4-carboxylic acid and 2-hydrazinyl-1,3-benzothiazole in a manner similar to general procedure 2.

¹H NMR (600 MHz, Acetone) δ 9.42 (br s, 1H), 8.95 (br s, 1H), 7.75 (dd, J=8.4, 7.8 Hz, 1H), 7.66 (d, J=7.8 Hz, 1H), 7.61-7.51 (m, 2H), 7.43 (d, J=7.8 Hz, 1H), 7.37 (dd, J=8.4, 2.4 Hz, 1H), 7.28-7.25 (m, 1H), 7.20 (t, J=54.0 Hz, 1H), 7.10-7.07 (m, 1H), 3.81 (s, 3H).

ESI-MS C₂₀H₁₄F₅N₅O₂S: 483.0788, found: 484.1 (M+H⁺)⁺.

HRMS (ESI, m/z): [M+H]⁺ calcd for C₂₀H₁₅F₅N₅O₂S: 484.0861; found: 484.0860.

### Example 35: 3-(difluoromethyl)-1-methyl-N-[4-(propan-2-yl)phenyl]-5-[3-(trifluoromethyl)phenoxy]-1H-pyrazole-4-carbohydrazide (Compound 35)

Reagents and conditions: [4-(propan-2-yl)phenyl]hydrazine hydrochloride, HATU, DIPEA, DMSO, 15 h, 72%.

Compound 35 was prepared using 3-(difluoromethyl)-1-methyl-5-[3-(trifluoromethyl)-phenoxy]-1H-pyrazole-4-carboxylic acid and [4-(propan-2-yl)phenyl]hydrazine hydrochloride in a manner similar to general procedure 3.

¹H NMR (600 MHz, Acetone) δ 8.81 (br s, 1H), 7.73 (dd, J=8.4, 7.8 Hz, 1H), 7.64 (d, J=7.8 Hz, 1H), 7.55 (s, 1H), 7.34 (dd, J=8.4, 2.4 Hz, 1H), 7.18 (t, J=54.0 Hz, 1H), 6.90 (br s, 1H), 6.87 (d, J=8.4 Hz, 2H), 6.43 (d, J=8.4 Hz, 2H), 3.80 (s, 3H), 2.73 (sep, J=7.1 Hz, 1H), 1.13 (d, J=7.1 Hz, 6H).

ESI-MS C₂₂H₂₁F₅N₄O₂: 468.1585, found: 467.0 (M−H⁺)⁻.

HRMS (ESI, m/z): [M+H]⁺ calcd for C₂₂H₂₂F₅N₄O₂: 469.1657; found: 469.1659.

### Example 36: N-cyclohexyl-3-(difluoromethyl)-1-methyl-5-[3-(trifluoromethyl)phenoxy]-1H-pyrazole-4-carbohydrazide (Compound 36)

Reagents and conditions: Cyclohexylhydrazine hydrochloride, HATU, DIPEA, DMSO, 15 h, 24%.

Compound 36 was prepared using 3-(difluoromethyl)-1-methyl-5-[3-(trifluoromethyl)-phenoxy]-1H-pyrazole-4-carboxylic acid and cyclohexylhydrazine hydrochloride in a manner similar to general procedure 3.

¹H NMR (600 MHz, Acetone) δ 8.35 (br s, 1H), 7.68 (dd, J=8.4, 7.8 Hz, 1H), 7.56 (dd, J=7.8, 0.6 Hz, 1H), 7.49 (s, 1H), 7.29 (dd, J=8.4, 2.4 Hz, 1H), 7.19 (t, J=54.0 Hz, 1H), 3.79 (s, 3H), 2.42-2.37 (m, 1H), 1.56-1.52 (m, 2H), 1.45-1.40 (m, 2H), 1.06-0.97 (m, 4H), 0.91-0.81 (m, 2H).

ESI-MS C₁₉H₂₁F₅N₄O₂: 432.1585, found: 433.2 (M+H⁺)⁺.

HRMS (ESI, m/z): [M+H]⁺ calcd for C₁₉H₂₂F₅N₄O₂: 433.1657; found: 433.1659.

### Example 37: N-(biphenyl-4-ylcarbonyl)-3-(difluoromethyl)-1-methyl-5-[3-(trifluoromethyl)phenoxy]-1H-pyrazole-4-carbohydrazide (Compound 37)

Reagents and conditions: Biphenyl-4-carbohydrazide, HATU, DIPEA, DMSO, 15 h, 57%.

Compound 37 was prepared using 3-(difluoromethyl)-1-methyl-5-[3-(trifluoromethyl)-phenoxy]-1H-pyrazole-4-carboxylic acid and biphenyl-4-carbohydrazide in a manner similar to general procedure 1.

¹H NMR (600 MHz, Acetone) δ 8.00 (d, J=8.1 Hz, 2H), 7.77 (d, J=8.1 Hz, 2H), 7.74-7.68 (m, 3H), 7.59 (d, J=7.8 Hz, 1H), 7.55 (s, 1H), 7.52-7.47 (m, 2H), 7.43-7.39 (m, 1H), 7.37 (d, J=8.4 Hz, 1H), 7.25 (t, J=54.0 Hz, 1H), 3.77 (s, 1H).

ESI-MS C₂₆H₁₉F₅N₄O₃: 530.1377, found: 529.0 (M−H⁺)⁻.

HRMS (ESI, m/z): [M+H]⁺ calcd for C₂₆H₂₀F₅N₄O₃: 531.1450; found: 531.1455.

### Example 38: 3-(difluoromethyl)-1-methyl-N-(morpholin-4-yl)-5-[3-(trifluoromethyl)phenoxy]-1H-pyrazole-4-carboxamide (Compound 38)

Reagents and conditions: Morpholin-4-amine, HATU, DIPEA, DMSO, 15 h, 69%.

Compound 38 was prepared using 3-(difluoromethyl)-1-methyl-5-[3-(trifluoromethyl)-phenoxy]-1H-pyrazole-4-carboxylic acid and morpholin-4-amine in a manner similar to general procedure 2.

¹H NMR (600 MHz, Acetone) δ 7.84 (br s, 1H), 7.68 (dd, J=8.4, 7.8 Hz, 1H), 7.56 (d, J=7.8 Hz, 1H), 7.50 (s, 1H), 7.33 (d, J=8.4 Hz, 1H), 7.11 (t, J=54.0 Hz, 1H), 3.79 (s, 3H), 3.56 (br s, 4H), 2.68-2.62 (m, 4H).

ESI-MS C₁₇H₁₇F₅N₄O₃: 420.1221, found: 421.1 (M+H⁺)⁺.

HRMS (ESI, m/z): [M+H]⁺ calcd for C₁₇H₁₈F₅N₄O₃: 421.1294; found: 421.1300.

### Example 39: 3-(difluoromethyl)-1-methyl-N-(4-methylpiperazin-1-yl)-5-[3-(trifluoromethyl)-phenoxy]-1H-pyrazole-4-carboxamide (Compound 39)

Reagents and conditions: 4-methylpiperazin-1-amine, HATU, DIPEA, DMSO, 15 h, 66%.

Compound 39 was prepared using 3-(difluoromethyl)-1-methyl-5-[3-(trifluoromethyl)-phenoxy]-1H-pyrazole-4-carboxylic acid and 4-methylpiperazin-1-amine in a manner similar to general procedure 2.

¹H NMR (600 MHz, Acetone) δ 7.70-7.64 (m, 2H), 7.56 (d, J=7.2 Hz, 1H), 7.50 (s, 1H), 7.32 (d, J=7.2 Hz, 1H), 7.12 (t, J=54.0 Hz, 1H), 3.78 (s, 3H), 2.63 (br s, 4H), 2.31 (br s, 4H), 2.14 (s, 3H).

ESI-MS C₁₈H₂₀F₅N₅O₂: 433.1537, found: 434.1 (M+H⁺)⁺.

HRMS (ESI, m/z): [M+H]⁺ calcd for C₁₈H₂₁F₅N₅O₂: 434.1610; found: 434.1615.

### Example 40: 3-(difluoromethyl)-1-methyl-N′-phenyl-5-[3-(trifluoromethyl)phenoxy]-1H-pyrazole-4-carbohydrazide (Compound 40)

Reagents and conditions: Phenylhydrazine hydrochloride, HATU, DIPEA, DMSO, 15 h, 56%.

Compound 40 was prepared using 3-(difluoromethyl)-1-methyl-5-[3-(trifluoromethyl)-phenoxy]-1H-pyrazole-4-carboxylic acid and phenylhydrazine hydrochloride in a manner similar to general procedure 3.

¹H NMR (600 MHz, Acetone) δ 8.83 (br s, 1H), 7.73 (dd, J=8.4, 7.8 Hz, 1H), 7.64 (d, J=7.8 Hz, 1H), 7.55 (s, 1H), 7.35 (dd, J=8.4, 2.4 Hz, 1H), 7.18 (t, J=54.0 Hz, 1H), 7.03 (br s, 1H), 6.98 (t, J=7.8 Hz, 2H), 6.68 (t, J=7.8 Hz, 1H), 6.49 (d, J=7.8 Hz, 2H), 3.81 (s, 3H).

ESI-MS C₁₉H₁₅F₅N₄O₂: 426.1115, found: 425.0 (M−H⁺)⁻.

HRMS (ESI, m/z): [M−H]⁻ calcd for C₁₉H₁₄F₅N₄O₂: 425.1042; found: 425.1049.

### Example 41: 3-(difluoromethyl)-1-methyl-N-(4-methylphenyl)-5-[3-(trifluoromethyl)phenoxy]-1H-pyrazole-4-carbohydrazide (Compound 41)

Reagents and conditions: (4-methylphenyl)hydrazine hydrochloride, HATU, DIPEA, DMSO, 15 h, 65%.

Compound 41 was prepared using 3-(difluoromethyl)-1-methyl-5-[3-(trifluoromethyl)-phenoxy]-1H-pyrazole-4-carboxylic acid and (4-methylphenyl)hydrazine hydrochloride in a manner similar to general procedure 3.

¹H NMR (600 MHz, Acetone) δ 8.80 (br s, 1H), 7.72 (dd, J=8.4, 7.8 Hz, 1H), 7.63 (d, J=7.8 Hz, 1H), 7.54 (s, 1H), 7.34 (d, J=8.4 Hz, 1H), 7.18 (t, J=54.0 Hz, 1H), 6.88 (br s, 1H), 6.79 (d, J=7.2 Hz, 2H), 6.40 (d, J=7.2 Hz, 2H), 3.80 (s, 3H), 2.14 (s, 3H).

ESI-MS C₂₀H₁₇F₅N₄O₂: 440.1272, found: 439.0 (M−H⁺)⁻.

HRMS (ESI, m/z): [M+H]⁺ calcd for C₂₀H₁₈F₅N₄O₂: 441.1344; found: 441.1343.

### Example 42: 3-(difluoromethyl)-N-(2,6-dimethylphenyl)-1-methyl-5-[3-(trifluoromethyl)-phenoxy]-1H-pyrazole-4-carbohydrazide (Compound 42)

Reagents and conditions: (2,6-dimethylphenyl)hydrazine hydrochloride, HATU, DIPEA, DMSO, 15 h, 66%.

Compound 42 was prepared using 3-(difluoromethyl)-1-methyl-5-[3-(trifluoromethyl)-phenoxy]-1H-pyrazole-4-carboxylic acid and (2,6-dimethylphenyl)hydrazine hydrochloride in a manner similar to general procedure 3.

¹H NMR (600 MHz, Acetone) δ 8.60 (br s, 1H), 7.64 (dd, J=8.4, 7.8 Hz, 1H), 7.56 (d, J=7.8 Hz, 1H), 7.49 (s, 1H), 7.23 (dd, J=8.4, 2.4 Hz, 1H), 7.18 (t, J=54.0 Hz, 1H), 6.83 (d, J=7.4 Hz, 2H), 6.74 (t, J=7.4 Hz, 1H), 6.39 (br t, J=3.6 Hz, 1H), 3.72 (s, 3H), 2.19 (s, 6H).

ESI-MS C₂₁H₁₉F₅N₄O₂: 454.1428, found: 453.1 (M+H⁺)⁺.

HRMS (ESI, m/z): [M−H]⁻ calcd for C₂₁H₁₈F₅N₄O₂: 453.1355; found: 453.1364.

### Example 43: N-(cyclopropylcarbonyl)-3-(difluoromethyl)-1-methyl-5-[3-(trifluoromethyl)-phenoxy]-1H-pyrazole-4-carbohydrazide (Compound 43)

Reagents and conditions: Cyclopropanecarbohydrazide, HATU, DIPEA, DMSO, 15 h, 62%.

Compound 43 was prepared using 3-(difluoromethyl)-1-methyl-5-[3-(trifluoromethyl)-phenoxy]-1H-pyrazole-4-carboxylic acid and cyclopropanecarbohydrazide in a manner similar to general procedure 2.

¹H NMR (600 MHz, Acetone) δ 7.67 (dd, J=8.4, 7.8 Hz, 1H), 7.56 (d, J=7.8 Hz, 1H), 7.50 (s, 1H), 7.31 (dd, J=8.4, 2.4 Hz, 1H), 7.16 (t, J=54.0 Hz, 1H), 3.74 (s, 3H), 1.69-1.63 (m, 1H), 0.80-0.74 (m, 2H), 0.75-0.69 (m, 2H).

ESI-MS C₁₇H₁₅F₅N₄O₃: 418.1064, found: 417.0 (M−H⁺)⁻.

HRMS (ESI, m/z): [M−H]⁻ calcd for C₁₇H₁₄F₅N₄O₃: 417.0992; found: 417.0996.

### Example 44: Ethyl 2-({3-(difluoromethyl)-1-methyl-5-[3-(trifluoromethyl)phenoxy]-1H-pyrazol-4-yl}carbonyl)hydrazinecarboxylate (Compound 44)

Reagents and conditions: Ethyl hydrazinecarboxylate, HATU, DIPEA, DMSO, 15 h, 57%.

Compound 44 was prepared using 3-(difluoromethyl)-1-methyl-5-[3-(trifluoromethyl)-phenoxy]-1H-pyrazole-4-carboxylic acid and ethyl hydrazinecarboxylate in a manner similar to general procedure 2.

¹H NMR (600 MHz, CDCl₃) δ 7.80 (br s, 1H), 7.53 (dd, J=8.4, 7.8 Hz, 1H), 7.48 (d, J=7.8 Hz, 1H), 7.30 (s, 1H), 7.09 (dd, J=8.4, 2.4 Hz, 1H), 7.07 (t, J=54.0 Hz, 1H), 6.52 (br s, 1H), 4.23-4.07 (m, 2H), 3.69 (s, 3H), 1.30-1.16 (m, 3H).

ESI-MS C₁₆H₁₅F₅N₄O₄: 422.1013, found: 423.1 (M+H⁺)⁺.

HRMS (ESI, m/z): [M+H]⁺ calcd for C₁₆H₁₆F₅N₄O₄: 423.1086; found: 423.1087.

### Example 45: 3-(difluoromethyl)-N-(2,4-difluorophenyl)-1-methyl-5-[3-(trifluoromethyl)-phenoxy]-1H-pyrazole-4-carbohydrazide (Compound 45)

Reagents and conditions: (2,4-difluorophenyl)hydrazine hydrochloride, HATU, DIPEA, DMSO, 15 h, 92%.

Compound 45 was prepared by mixing 3-(difluoromethyl)-1-methyl-5-[3-(trifluoromethyl)-phenoxy]-1H-pyrazole-4-carboxylic acid (1 g, 3 mmol, 1 eq.), HATU (1.5 g, 3.9 mmol, 1.3 eq.) and DIPEA (1.5 g, 11.9 mmol, 4 eq.) in DMSO (15 mL) for 1 h at room temperature. (2,4-difluorophenyl)hydrazine hydrochloride (0.8 g, 5.9 mmol, 2 eq.) was added to the reaction mixture and was stirred at room temperature for 15 h. The residue was purified by flash chromatography to give Compound 45 (1.26 g, 92%).

¹H NMR (600 MHz, Acetone) δ 8.99 (br s, 1H), 7.73 (dd, J=8.4, 7.8 Hz, 1H), 7.65 (d, J=7.8 Hz, 1H), 7.55 (s, 1H), 7.36 (dd, J=8.4, 2.4 Hz, 1H), 7.16 (t, J=54.0 Hz, 1H), 6.96 (br s, 1H), 6.90 (ddd, J=11.7, 8.7, 2.7 Hz, 1H), 6.57-6.51 (m, 1H), 6.25 (td, J=9.3, 5.6 Hz, 1H), 3.81 (s, 3H).

ESI-MS C₁₉H₁₃F₇N₄O₂: 462.0927, found: 461.0 (M−H⁺)⁻.

HRMS (ESI, m/z): [M+H]⁺ calcd for C₁₉H₁₄F₇N₄O₂: 463.0999; found: 463.0996.

### Example 46: 3-(difluoromethyl)-N-(4-fluorophenyl)-1-methyl-5-[3-(trifluoromethyl)phenoxy]-1H-pyrazole-4-carbohydrazide (Compound 46)

Reagents and conditions: (4-fluorophenyl)hydrazine hydrochloride, HATU, DIPEA, DMSO, 15 h, 32%.

Compound 46 was prepared using 3-(difluoromethyl)-1-methyl-5-[3-(trifluoromethyl)-phenoxy]-1H-pyrazole-4-carboxylic acid and (4-fluorophenyl)hydrazine hydrochloride in a manner similar to general procedure 3.

¹H NMR (600 MHz, Acetone) δ 8.89 (br s, 1H), 7.73 (dd, J=8.4, 7.8 Hz, 1H), 7.65 (d, J=7.8 Hz, 1H), 7.55 (s, 1H), 7.35 (dd, J=8.4, 2.4 Hz, 2H), 7.17 (t, J=54.0 Hz, 1H), 7.05 (br s, 1H), 6.78-6.72 (m, 2H), 6.51-6.46 (m, 2H), 3.81 (s, 3H).

ESI-MS C₁₉H₁₄F₆N₄O₂: 444.1021, found: 443.0 (M−H⁺)⁻.

HRMS (ESI, m/z): [M+H]⁺ calcd for C₁₉H₁₅F₆N₄O₂: 445.1094; found: 445.1092.

### Example 47: 3-(difluoromethyl)-1-methyl-5-[3-(trifluoromethyl)phenoxy]-N-[4-(trifluoromethyl)phenyl]-1H-pyrazole-4-carbohydrazide (Compound 47)

Reagents and conditions: [4-(trifluoromethyl)phenyl]hydrazine, HATU, DIPEA, DMSO, 15 h, 12%.

Compound 47 was prepared using 3-(difluoromethyl)-1-methyl-5-[3-(trifluoromethyl)-phenoxy]-1H-pyrazole-4-carboxylic acid and [4-(trifluoromethyl)phenyl]hydrazine in a manner similar to general procedure 1.

¹H NMR (600 MHz, Acetone) δ 8.99 (br s, 1H), 7.75 (dd, J=8.4, 7.8 Hz, 1H), 7.66 (d, J=7.8 Hz, 1H), 7.60 (br s, 1H), 7.56 (s, 1H), 7.38 (dd, J=8.4, 2.4 Hz, 1H), 7.30 (d, J=8.7 Hz, 2H), 7.16 (t, J=54.0 Hz, 1H), 6.60 (d, J=8.7 Hz, 2H), 3.82 (s, 3H).

ESI-MS C₂₀H₁₄F₈N₄O₂: 494.0989, found: 493.0 (M−H⁺)⁻.

HRMS (ESI, m/z): [M+H]⁺ calcd for C₂₀H₁₅F₈N₄O₂: 495.1062; found: 495.1058.

### Example 48: N-(4-chloro-2-methylphenyl)-3-(difluoromethyl)-1-methyl-5-[3-(trifluoromethyl)phenoxy]-1H-pyrazole-4-carbohydrazide (Compound 48)

Reagents and conditions: (4-chloro-2-methylphenyl)hydrazine hydrochloride, HATU, DIPEA, DMSO, 15 h, 37%.

Compound 48 was prepared using 3-(difluoromethyl)-1-methyl-5-[3-(trifluoromethyl)-phenoxy]-1H-pyrazole-4-carboxylic acid and (4-chloro-2-methylphenyl)hydrazine hydrochloride in a manner similar to general procedure 3.

¹H NMR (600 MHz, Acetone) δ 8.92 (br s, 1H), 7.74 (dd, J=8.4, 7.8 Hz, 1H), 7.66 (d, J=7.8 Hz, 1H), 7.55 (s, 1H), 7.36 (dd, J=8.4, 2.4 Hz, 1H), 7.17 (t, J=54.0 Hz, 1H), 7.01 (d, J=1.8 Hz, 1H), 6.75-6.68 (m, 2H), 6.10 (d, J=9.0 Hz, 1H), 3.81 (s, 3H), 2.19 (s, 3H).

ESI-MS C₂₀H₁₆ClF₅N₄O₂: 474.0882, found: 473.0 (M−H⁺)⁻.

HRMS (ESI, m/z): [M+H]⁺ calcd for C₂₀H₁₇ClF₅N₄O₂: 475.0955; found: 475.0954.

### Example 49: N-tert-butyl-3-(difluoromethyl)-1-methyl-5-[3-(trifluoromethyl)phenoxy]-1H-pyrazole-4-carbohydrazide (Compound 49)

Reagents and conditions: tert-butylhydrazine hydrochloride, HATU, DIPEA, DMSO, 15 h, 40%.

Compound 49 was prepared using 3-(difluoromethyl)-1-methyl-5-[3-(trifluoromethyl)-phenoxy]-1H-pyrazole-4-carboxylic acid and tert-butylhydrazine hydrochloride in a manner similar to general procedure 3.

¹H NMR (600 MHz, Acetone) δ 8.22 (br s, 1H), 7.68 (dd, J=8.4, 7.8 Hz, 1H), 7.56 (d, J=7.8 Hz, 1H), 7.48 (s, 1H), 7.30 (dd, J=8.4, 2.4 Hz, 2H), 7.18 (t, J=54.0 Hz, 1H), 3.79 (s, 3H), 0.80 (s, 9H).

ESI-MS C₁₇H₁₉F₅N₄O₂: 406.1428, found: 407.2 (M+H⁺)⁺.

HRMS (ESI, m/z): [M+H]⁺ calcd for C₁₇H₂₀F₅N₄O₂: 407.1501; found: 407.1499.

### Example 50: 3-(difluoromethyl)-1-methyl-N-(thiophen-2-ylmethyl)-5-[3-(trifluoromethyl)-benzyl]-1H-pyrazole-4-carboxamide (Compound 50)

Reagents and conditions: 1-(thiophen-2-yl)methanamine, HATU, DIPEA, DMSO, 15 h, 33%.

Compound 50 was prepared using 3-(difluoromethyl)-1-methyl-5-[3-(trifluoromethyl)-phenoxy]-1H-pyrazole-4-carboxylic acid and 1-(thiophen-2-yl)methanamine in a manner similar to general procedure 1.

¹H NMR (600 MHz, Acetone) δ 7.62 (dd, J=8.4, 7.8 Hz, 1H), 7.54 (dd, J=7.8, 1.2 Hz, 1H), 7.48 (br s, 1H), 7.42 (s, 1H), 7.27 (t, J=54.0 Hz, 1H), 7.22 (dd, J=8.4, 1.8 Hz, 1H), 7.20-7.18 (m, 1H), 6.86-6.80 (m, 1H), 6.80-6.75 (m, 1H), 4.56 (d, J=6.0 Hz, 2H), 3.73 (s, 3H).

ESI-MS C₁₈H₁₄F₅N₃O₂S: 431.0727, found: 432.1 (M+H⁺)⁺.

HRMS (ESI, m/z): [M+H]⁺ calcd for C₁₈H₁₅F₅N₃O₂S: 432.0800; found: 432.0803.

### Example 51: 3-(difluoromethyl)-1-methyl-N-[2-(thiophen-3-yl)ethyl]-5-[3-(trifluoromethyl)-phenoxy]-1H-pyrazole-4-carboxamide (Compound 51)

Reagents and conditions: 2-(thiophen-3-yl)ethanamine, HATU, DIPEA, DMSO, 15 h, 37%.

Compound 51 was prepared using 3-(difluoromethyl)-1-methyl-5-[3-(trifluoromethyl)-phenoxy]-1H-pyrazole-4-carboxylic acid and 2-(thiophen-3-yl)ethanamine in a manner similar to general procedure 1.

¹H NMR (600 MHz, Acetone) δ 7.67 (dd, J=8.4, 7.8 Hz, 1H), 7.58 (dd, J=7.8, 0.6 Hz, 1H), 7.43 (s, 1H), 7.29 (dd, J=5.1, 3.0 Hz, 1H), 7.25 (t, J=54.0 Hz, 1H), 7.23 (dd, J=8.4, 2.4 Hz, 1H), 7.01-6.95 (m, 1H), 6.92 (br s, 1H), 6.87 (dd, J=5.1, 1.2 Hz, 1H), 3.71 (s, 3H), 3.49-3.43 (m, 2H), 2.69 (t, J=6.9 Hz, 2H).

ESI-MS C₁₉H₁₆F₅N₃O₂S: 445.0883, found: 446.2 (M+H⁺)⁺.

HRMS (ESI, m/z): [M+H]⁺ calcd for C₁₉H₁₇F₅N₃O₂S: 446.0956; found: 446.0956.

### Example 52: N-(azepan-1-yl)-3-(difluoromethyl)-1-methyl-5-[3-(trifluoromethyl)phenoxy]-1H-pyrazole-4-carboxamide (Compound 52)

Reagents and conditions: Azepan-1-amine, 50% T₃P, DIPEA, DMF, 15 h, 34%.

Compound 52 was prepared using 3-(difluoromethyl)-1-methyl-5-[3-(trifluoromethyl)-phenoxy]-1H-pyrazole-4-carboxylic acid and azepan-1-amine in a manner similar to general procedure 4.

¹H NMR (600 MHz, Acetone) δ 8.08 (br s, 1H), 7.68 (dd, J=8.4, 7.8 Hz, 1H), 7.57 (d, J=7.8 Hz, 1H), 7.50 (s, 1H), 7.32 (d, J=8.4 Hz, 1H), 7.13 (t, J=54.0 Hz, 1H), 3.78 (s, 3H), 2.84-2.82 (m, 4H), 1.51 (s, 8H).

ESI-MS C₁₉H₂₁F₅N₄O₂: 432.1585, found: 433.2 (M+H⁺)⁺.

HRMS (ESI, m/z): [M+H]⁺ calcd for C₁₉H₂₂F₅N₄O₂: 433.1657; found: 433.1657.

### Example 53: N-(cyclopentylmethyl)-3-(difluoromethyl)-1-methyl-5-[3-(trifluoromethyl)-phenoxy]-1H-pyrazole-4-carboxamide (Compound 53)

Reagents and conditions: 1-cyclopentylmethanamine, HATU, DIPEA, DMSO, 15 h, 56%.

Compound 53 was prepared using 3-(difluoromethyl)-1-methyl-5-[3-(trifluoromethyl)-phenoxy]-1H-pyrazole-4-carboxylic acid and 1-cyclopentylmethanamine in a manner similar to general procedure 1.

¹H NMR (600 MHz, Acetone) δ 7.69 (dd, J=8.4, 7.8 Hz, 1H), 7.57 (dd, J=7.8, 0.6 Hz, 1H), 7.49 (s, 1H), 7.27 (dd, J=8.4, 2.4 Hz, 1H), 7.24 (t, J=54.0 Hz, 1H), 6.85 (br s, 1H), 3.76 (s, 3H), 3.13 (t, J=6.6 Hz, 2H), 1.87 (quin, J=7.4 Hz, 1H), 1.52-1.35 (m, 6H), 1.06-0.94 (m, 2H).

ESI-MS C₁₉H₂₀F₅N₃O₂: 417.1476, found: 418.1 (M+H⁺)⁺.

HRMS (ESI, m/z): [M+H]⁺ calcd for C₁₉H₂₁F₅N₃O₂: 418.1548; found: 418.1554.

### Example 54: 3-(difluoromethyl)-N-(hexahydrocyclopenta[c]pyrrol-2(1H)-yl)-1-methyl-5-[3-(trifluoromethyl)phenoxy]-1H-pyrazole-4-carboxamide (Compound 54)

Reagents and conditions: Hexahydrocyclopenta[c]pyrrol-2(1H)-amine hydrochloride, HATU, DIPEA, DMSO, 15 h, 42%.

Compound 54 was prepared using 3-(difluoromethyl)-1-methyl-5-[3-(trifluoromethyl)-phenoxy]-1H-pyrazole-4-carboxylic acid and hexahydrocyclopenta[c]pyrrol-2(1H)-amine hydrochloride in a manner similar to general procedure 3.

¹H NMR (600 MHz, CDCl₃) δ 7.50 (t, J=7.5 Hz, 1H), 7.46 (d, J=7.5 Hz, 1H), 7.29 (s, 1H), 7.10-7.04 (m, 1H), 7.07 (t, J=54.0 Hz, 1H), 6.56 (br s, 1H), 3.72 (s, 3H), 2.98 (t, J=8.1 Hz, 2H), 2.56 (br s, 2H), 2.25 (dd, J=8.4, 5.4 Hz, 2H), 1.64-1.60 (m, 3H), 1.53-1.48 (m, 1H), 1.44-1.36 (m, 2H).

ESI-MS C₂₀H₂₁F₅N₄O₂: 444.1585, found: 445.1 (M+H⁺)⁺.

HRMS (ESI, m/z): [M+H]⁺ calcd for C₂₀H₂₂F₅N₄O₂: 445.1657; found: 445.1653.

### Example 55: Methyl 3-[({3-(difluoromethyl)-1-methyl-5-[3-(trifluoromethyl)phenoxy]-1H-pyrazol-4-yl}carbonyl)amino]thiophene-2-carboxylate (Compound 55)

Reagents and conditions: Methyl 3-aminothiophene-2-carboxylate, HATU, DIPEA, DMSO, 70° C., 15 h, 15%.

Compound 55 was prepared using 3-(difluoromethyl)-1-methyl-5-[3-(trifluoromethyl)-phenoxy]-1H-pyrazole-4-carboxylic acid and methyl 3-aminothiophene-2-carboxylate in a manner similar to general procedure 1 except heating at 70° C.

¹H NMR (600 MHz, Acetone) δ 8.14 (d, J=5.4 Hz, 1H), 7.77 (d, J=5.4 Hz, 1H), 7.68-7.63 (m, 2H), 7.54 (dd, J=7.8, 0.6 Hz, 1H), 7.33 (t, J=54.0 Hz, 1H), 7.31 (dd, J=8.4, 2.4 Hz, 1H), 3.87 (s, 3H), 3.79 (s, 3H).

ESI-MS C₁₉H₁₄F₅N₃O₄S: 475.0625, found: 476.1 (M+H⁺)⁺.

HRMS (ESI, m/z): [M+H]⁺ calcd for C₁₉H₁₅F₅N₃O₄S: 476.0698; found: 476.0697.

### Example 56: Methyl 2-[({3-(difluoromethyl)-1-methyl-5-[3-(trifluoromethyl)phenoxy]-1H-pyrazol-4-yl}carbonyl)amino]thiophene-3-carboxylate (Compound 56)

Reagents and conditions: Methyl 2-aminothiophene-3-carboxylate, 50% T₃P, DIPEA, DMF, 70° C., 15 h, 35%.

Compound 56 was prepared using 3-(difluoromethyl)-1-methyl-5-[3-(trifluoromethyl)-phenoxy]-1H-pyrazole-4-carboxylic acid and methyl 2-aminothiophene-3-carboxylate in a manner similar to general procedure 4 except heating at 70° C.

¹H NMR (600 MHz, Acetone) δ 7.73 (s, 1H), 7.66 (dd, J=8.4, 7.8 Hz, 1H), 7.56 (dd, J=7.8, 0.6 Hz, 1H), 7.33 (t, J=53.7 Hz, 1H), 7.32 (dd, J=8.4, 2.4 Hz, 1H), 7.17 (d, J=5.7 Hz, 1H), 6.94 (dd, J=5.7, 0.6 Hz, 1H), 3.86 (s, 3H), 3.80 (s, 3H).

ESI-MS C₁₉H₁₄F₅N₃O₄S: 475.0625, found: 474.0 (M−H⁺)⁻.

HRMS (ESI, m/z): [M+H]⁺ calcd for C₁₉H₁₅F₅N₃O₄S: 476.0698; found: 476.0691.

### Example 57: N-(2-carbamoylthiophen-3-yl)-3-(difluoromethyl)-1-methyl-5-[3-(trifluoromethyl)phenoxy]-1H-pyrazole-4-carboxamide (Compound 57)

Reagents and conditions: 3-aminothiophene-2-carboxamide, HATU, DIPEA, DMSO, 70° C., 15 h, 54%.

Compound 57 was prepared using 3-(difluoromethyl)-1-methyl-5-[3-(trifluoromethyl)-phenoxy]-1H-pyrazole-4-carboxylic acid and 3-aminothiophene-2-carboxamide in a manner similar to general procedure 1 except heating at 70° C.

¹H NMR (600 MHz, Acetone) δ 8.12 (d, J=5.7 Hz, 1H), 7.64-7.60 (m, 2H), 7.58 (d, J=5.7 Hz, 1H), 7.52-7.48 (m, 1H), 7.33 (t, J=54.0 Hz, 1H), 7.30 (dd, J=8.4, 2.4 Hz, 1H), 3.79 (s, 3H).

ESI-MS C₁₈H₁₃F₅N₄O₃S: 460.0629, found: 459.0 (M−H⁺)⁻.

HRMS (ESI, m/z): [M−H]⁻ calcd for C₁₈H₁₂F₅N₄O₃S: 459.0556; found: 459.0555.

### Example 58: 3-(difluoromethyl)-1-methyl-N-[1-methyl-3-(thiophen-2-yl)-1H-pyrazol-5-yl]-5-[3-(trifluoromethyl)phenoxy]-1H-pyrazole-4-carboxamide (Compound 58)

Reagents and conditions: 1-methyl-3-(thiophen-2-yl)-1H-pyrazol-5-amine, HATU, DIPEA, DMSO, 70° C., 15 h, 37%.

Compound 58 was prepared using 3-(difluoromethyl)-1-methyl-5-[3-(trifluoromethyl)-phenoxy]-1H-pyrazole-4-carboxylic acid and 1-methyl-3-(thiophen-2-yl)-1H-pyrazol-5-amine in a manner similar to general procedure 1 except heating at 70° C.

¹H NMR (600 MHz, Acetone) δ 8.86 (br s, 1H), 7.72 (dd, J=8.4, 7.8 Hz, 1H), 7.61 (dd, J=7.8, 0.6 Hz, 1H), 7.58 (s, 1H), 7.43 (dd, J=8.4, 2.4 Hz, 1H), 7.34-7.30 (m, 1H), 7.31-7.27 (m, 1H), 7.20 (t, J=54.0 Hz, 1H), 7.04-7.01 (m, 1H), 6.39 (s, 1H), 3.82 (s, 3H), 3.47 (s, 3H).

ESI-MS C₂₁H₁₆F₅N₅O₂S: 497.0945, found: 498.1 (M+H⁺)⁺.

HRMS (ESI, m/z): [M+H]⁺ calcd for C₂₁H₁₇F₅N₅O₂S: 498.1018; found: 498.1025.

### Example 59: Ethyl 2-[({3-(difluoromethyl)-1-methyl-5-[3-(trifluoromethyl)phenoxy]-1H-pyrazol-4-yl}carbonyl)amino]-4,5-dimethylthiophene-3-carboxylate (Compound 59)

Reagents and conditions: Ethyl 2-amino-4,5-dimethylthiophene-3-carboxylate, HATU, DIPEA, DMSO, 70° C., 15 h, 15%.

Compound 59 was prepared using 3-(difluoromethyl)-1-methyl-5-[3-(trifluoromethyl)-phenoxy]-1H-pyrazole-4-carboxylic acid and ethyl 2-amino-4,5-dimethylthiophene-3-carboxylate in a manner similar to general procedure 1 except heating at 70° C.

¹H NMR (600 MHz, Acetone) δ 7.68-7.63 (m, 2H), 7.55 (dd, J=7.8, 0.6 Hz, 1H), 7.33 (t, J=54.0 Hz, 1H), 7.30 (dd, J=8.4, 2.4 Hz, 1H), 4.31 (q, J=7.1 Hz, 2H), 3.77 (s, 3H), 2.23 (s, 3H), 2.20 (s, 3H), 1.33 (t, J=7.1 Hz, 3H).

ESI-MS C₂₂H₂₀F₅N₃O₄S: 517.1095, found: 516.0 (M−H⁺)⁻.

HRMS (ESI, m/z): [M+H]⁺ calcd for C₂₂H₂₁F₅N₃O₄S: 518.1167; found: 518.1160.

### Example 60: N-({3-(difluoromethyl)-1-methyl-5-[3-(trifluoromethyl)phenoxy]-1H-pyrazol-4-yl}carbonyl)-5-methyl-1,2-oxazole-3-carbohydrazide (Compound 60)

Reagents and conditions: 5-methyl-1,2-oxazole-3-carbohydrazide, HATU, DIPEA, DMSO, 15 h, 62%.

Compound 60 was prepared using 3-(difluoromethyl)-1-methyl-5-[3-trifluoromethyl)-phenoxy]-1H-pyrazole-4-carboxylic acid and 5-methyl-1,2-oxazole-3-carbohydrazide in a manner similar to general procedure 1.

¹H NMR (600 MHz, Acetone) δ 7.69 (dd, J=8.4, 7.8 Hz, 1H), 7.58 (d, J=7.8 Hz, 1H), 7.52 (s, 1H), 7.33 (dd, J=8.4, 2.4 Hz, 1H), 7.22 (t, J=54.0 Hz, 1H), 6.46 (s, 1H), 3.75 (s, 3H), 2.48 (s, 3H).

ESI-MS C₁₈H₁₄F₅N₅O₄: 459.0966, found: 460.1 (M+H⁺)⁺.

HRMS (ESI, m/z): [M+Na]⁺ calcd for C₁₈H₁₄F₅N₅O₄Na: 482.0858; found: 482.0854.

### Example 61: N-({3-(difluoromethyl)-1-methyl-5-[3-(trifluoromethyl)phenoxy]-1H-pyrazol-4-yl}carbonyl)-1,3-thiazole-2-carbohydrazide (Compound 61)

Reagents and conditions: 1,3-thiazole-2-carbohydrazide, HATU, DIPEA, DMSO, 15 h, 60%.

Compound 61 was prepared using 3-(difluoromethyl)-1-methyl-5-[3-(trifluoromethyl)-phenoxy]-1H-pyrazole-4-carboxylic acid and 1,3-thiazole-2-carbohydrazide in a manner similar to general procedure 1.

¹H NMR (600 MHz, Acetone) δ 7.97 (d, J=3.0 Hz, 1H), 7.96 (d, J=3.0 Hz, 1H), 7.69 (dd, J=8.4, 7.8 Hz, 1H), 7.58 (d, J=7.8 Hz, 1H), 7.53 (s, 1H), 7.34 (dd, J=8.4, 2.4 Hz, 1H), 7.23 (t, J=54.0 Hz, 1H), 3.76 (s, 3H).

ESI-MS C₁₇H₁₂F₅N₅O₃S: 461.0581, found: 462.1 (M+H⁺)⁺.

HRMS (ESI, m/z): [M+H]⁺ calcd for C₁₇H₁₃F₅N₅O₃S: 462.0654; found: 462.0660.

### Example 62: N-({3-(difluoromethyl)-1-methyl-5-[3-(trifluoromethyl)phenoxy]-1H-pyrazol-4-yl}carbonyl)pyrazine-2-carbohydrazide (Compound 62)

Reagents and conditions: Pyrazine-2-carbohydrazide, HATU, DIPEA, DMSO, 15 h, 54%.

Compound 62 was prepared using 3-(difluoromethyl)-1-methyl-5-[3-(trifluoromethyl)-phenoxy]-1H-pyrazole-4-carboxylic acid and pyrazine-2-carbohydrazide in a manner similar to general procedure 1.

¹H NMR (600 MHz, Acetone) δ 9.17 (d, J=1.8 Hz, 1H), 8.86 (d, J=2.4 Hz, 1H), 8.66 (dd, J=2.4, 1.8 Hz, 1H), 7.70 (dd, J=8.4, 7.8 Hz, 1H), 7.58 (d, J=7.8 Hz, 1H), 7.54 (s, 1H), 7.36 (dd, J=8.4, 2.4 Hz, 1H), 7.23 (t, J=54.0 Hz, 1H), 3.77 (s, 3H).

ESI-MS C₁₈H₁₃F₅N₆O₃: 456.0969, found: 457.1 (M+H⁺)⁺.

HRMS (ESI, m/z): [M+H]⁺ calcd for C₁₈H₁₄F₅N₆O₃: 457.1042; found: 457.1041.

### Example 63: N-({3-(difluoromethyl)-1-methyl-5-[3-(trifluoromethyl)phenoxy]-1H-pyrazol-4-yl}carbonyl)pyrimidine-4-carbohydrazide (Compound 63)

Reagents and conditions: Pyrimidine-4-carbohydrazide, HATU, DIPEA, DMSO, 15 h, 56%.

Compound 63 was prepared using 3-(difluoromethyl)-1-methyl-5-[3-(trifluoromethyl)-phenoxy]-1H-pyrazole-4-carboxylic acid and pyrimidine-4-carbohydrazide in a manner similar to general procedure 1.

¹H NMR (600 MHz, Acetone) δ 9.24 (d, J=1.2 Hz, 1H), 9.07 (d, J=4.8 Hz, 1H), 8.02 (dd, J=4.8, 1.2 Hz, 1H), 7.70 (dd, J=8.4, 7.8 Hz, 1H), 7.58 (d, J=7.8 Hz, 1H), 7.54 (s, 1H), 7.36 (dd, J=8.4, 2.4 Hz, 2H), 7.22 (t, J=54.0 Hz, 1H), 3.77 (d, J=3.0 Hz, 3H).

ESI-MS C₁₈H₁₃F₅N₆O₃: 456.0969, found: 457.1 (M+H⁺)⁺.

HRMS (ESI, m/z): [M+H]⁺ calcd for C₁₈H₁₄F₅N₆O₃: 457.1042; found: 457.1039.

### Example 64: 3-(difluoromethyl)-1-methyl-N-[2-(1H-pyrrol-1-yl)phenyl]-5-[3-(trifluoromethyl)phenoxy]-1H-pyrazole-4-carboxamide (Compound 64)

Reagents and conditions: 2-(1H-pyrrol-1-yl)aniline, 50% T₃P, DIPEA, DMF, 70° C., 15 h, 26%.

Compound 64 was prepared using 3-(difluoromethyl)-1-methyl-5-[3-(trifluoromethyl)-phenoxy]-1H-pyrazole-4-carboxylic acid and 2-(1H-pyrrol-1-yl)aniline in a manner similar to general procedure 4 except heating at 70° C.

¹H NMR (600 MHz, Acetone) δ 8.33 (d, J=8.4 Hz, 1H), 8.20 (br s, 1H), 7.65 (dd, J=8.4, 7.8 Hz, 1H), 7.56 (dd, J=7.8, 0.6 Hz, 1H), 7.40-7.35 (m, 1H), 7.32 (s, 1H), 7.28 (t, J=53.7 Hz, 1H), 7.25-7.17 (m, 2H), 7.16 (dd, J=8.4, 1.8 Hz, 1H), 6.85-6.76 (m, 2H), 6.27-6.18 (m, 2H), 3.65 (s, 3H).

ESI-MS C₂₃H₁₇F₅N₄O₂: 476.1272, found: 475.0 (M−H⁺)⁻.

HRMS (ESI, m/z): [M−H]⁻ calcd for C₂₃H₁₆F₅N₄O₂: 475.1199; found: 475.1202.

### Example 65: 3-(difluoromethyl)-N-(1H-indazol-5-yl)-1-methyl-5-[3-(trifluoromethyl)phenoxy]-1H-pyrazole-4-carboxamide (Compound 65)

Reagents and conditions: 1H-indazol-5-amine, TBTU, DIPEA, DMSO, 70° C., 15 h, 85%.

Compound 65 was prepared by mixing 3-(difluoromethyl)-1-methyl-5-[3-(trifluoromethyl)-phenoxy]-1H-pyrazole-4-carboxylic acid (1 g, 3 mmol, 1 eq.), TBTU (1.2 g, 3.9 mmol, 1.3 eq.) and DIPEA (0.6 g, 4.8 mmol, 1.5 eq.) were stirred in DMSO (15 mL) for 1 h at room temperature. 1H-indazol-5-amine (0.8 g, 5.9 mmol, 2 eq.) was added to the reaction mixture and was stirred at 70° C. for 15 h. The residue was purified by flash chromatography to give Compound 65 (1.15 g, 85%).

¹H NMR (600 MHz, Acetone) δ 8.76 (br s, 1H), 8.02 (d, J=1.8 Hz, 1H), 7.96 (d, J=1.2 Hz, 1H), 7.69-7.63 (m, 1H), 7.60 (s, 1H), 7.55-7.50 (m, 1H), 7.47-7.44 (m, 1H), 7.41 (dd, J=8.4, 2.4 Hz, 1H), 7.32 (dd, J=9.0, 2.1 Hz, 1H), 7.22 (t, J=54.0 Hz, 1H), 3.82 (s, 3H) ESI-MS C₂₀H₁₄F₅N₅O₂: 451.1068, found: 452.1 (M+H⁺)⁺.

HRMS (ESI, m/z): [M+H]⁺ calcd for C₂₀H₁₅F₅N₅O₂: 452.1140; found: 452.1137.

### Example 66: 3-(difluoromethyl)-1-methyl-N-(thieno[3,2-b]thiophen-2-ylcarbonyl)-5-[3-(trifluoromethyl)phenoxy]-1H-pyrazole-4-carbohydrazide (Compound 66)

Reagents and conditions: (1) tert-butyl hydrazinecarboxylate, HATU, DIPEA, DMSO, 15 h, 107%. (2) TFA, DCM, 15 h, 40%. (3) Thieno[3,2-b]thiophene-2-carboxylic acid, HATU, DIPEA, DMSO, 15 h, 18%.

### tert-Butyl 2-({3-(difluoromethyl)-1-methyl-5-[3-(trifluoromethyl)phenoxy]-1H-pyrazol-4-yl}carbonyl)hydrazinecarboxylate (Compound 66.1)

3-(difluoromethyl)-1-methyl-5-[3-(trifluoromethyl)phenoxy]-1H-pyrazole-4-carboxylic acid (600 mg, 1.8 mmol, 1 eq.), HATU (882.1 mg, 2.3 mmol, 1.3 eq.) and DIPEA (80.7 mg, 6.2 mmol, 3.5 eq.) were stirred in DMSO (9 mL) for 1 h at room temperature. tert-butyl hydrazinecarboxylate (471.7 mg, 3.6 mmol, 2 eq.) was added to the reaction mixture and was stirred at room temperature for 15 h. The residue was purified by HPLC RP-C18 column to give 72.1 (858.1 mg, 107%)

### 3-(Difluoromethyl)-1-methyl-5-[3-(trifluoromethyl)phenoxy]-1H-pyrazole-4-carbohydrazide (Compound 66.2)

To a solution of 73.1 (858.1 mg, 1.9 mmol) in CH₂Cl₂ (19 mL) was added TFA (19 mL). The reaction mixture was stirred at room temperature for overnight. After the reaction was completed, the excess of TFA was removed under vacuum. The residue was purified by flash chromatography to give 73.2 (265.7 mg, 40%)

### Compound 66

Thieno[3,2-b]thiophene-2-carboxylic acid (63.1 mg, 0.3 mmol, 2 eq.), HATU (169.4 mg, 0.4 mmol, 2.6 eq.) and DIPEA (77.5 mg, 0.6 mmol, 3.5 eq.) were stirred in DMSO (1 mL) for 1 h at room temperature. 73.2 (60 mg, 0.2 mmol, 1 eq.) was added to the reaction mixture and was stirred at room temperature for 15 h. The residue was purified by HPLC RP-C18 column to give Compound 66 (16.3 mg, 18%).

¹H NMR (600 MHz, Acetone) δ 8.10 (s, 1H), 7.79 (d, J=4.8 Hz, 1H), 7.70 (dd, J=8.4, 7.8 Hz, 1H), 7.59 (d, J=7.8 Hz, 1H), 7.53 (s, 1H), 7.46 (d, J=4.8 Hz, 1H), 7.34 (dd, J=8.4, 2.4 Hz, 1H), 7.23 (t, J=54.0 Hz, 1H), 3.76 (s, 3H).

ESI-MS C₂₀H₁₃F₅N₄O₃S₂: 516.0349, found: 517.1 (M+H⁺)⁺.

HRMS (ESI, m/z): [M+H]⁺ calcd for C₂₀H₁₄F₅N₄O₃S₂: 517.0422; found: 517.0423.

### Eu-GTP Assays

Eu-GTP assays were performed to study G protein-coupled receptors (GPCRs) and their interactions with G proteins. The assay reported the activation of GPCRs by quantifying the exchange of guanosine diphosphate (GDP) for guanosine triphosphate (GTP) on the G protein. When a ligand binds to a EP4, it activates the associated G protein by promoting the exchange of GDP for GTP on the G protein's alpha subunit. The assay used a europium-labeled GTP analog, which emits a luminescent signal when the GTP is bound to the G protein. The intensity of the signal correlated with the amount of activated G protein. Membranes from cells expressing the EP4 were used, then addition of the indicated compound was followed at various concentration. Following stimulation, the europium-labeled GTP was introduced, allowing it to bind to the activated G protein.

The luminescence signal was measured, and the intensity indicated the level of GPCR activation with the confirmation of ligand association with EP4.

Compounds 3-5, 8, 10, 15, 20, 24, 27, 28, 32, 35, and 45 were evaluated. Each showed effective activation of G protein over a broad range of concentration, as compared to E7046.

### Secresome Analysis

The compounds of this invention induce release of secresomes containing proteins, lipids, and sometimes nucleic acids. Some secresomes also contain enzymes or other molecules that are secreted from cells.

Six compounds of this invention (i.e., Compounds 8, 12, 28, 45, 54, and 66) were evaluated for their induction effectiveness following known procedures, see, e.g., Chen et al., Stem Cells Translational Medicine 8, 707-723 (2019); Li, U.S. Pat. No. 11,149,277; and Bender et al., Am. J. Transl. Res. 5, 92-102 (2013). Compound E7046 was used as a control (see Jiao et al., J. Biomed. Sci. 30, 62 (2023); and Francica et al., Cancer Research Communications 3, 1486-1500 (2023)).

Stem cells were cultured in a medium containing a compound of this invention for a predetermined period of time (e.g., 4 days). The culture medium was collected, which contained the stem cells and the secresomes released from the stem cells. The medium was subjected to Bradford assay for protein concentration. The number of vesicles in the medium was measured using CytoFLEX Flow Cytometer.

The results show that the six compounds of this invention each induced release of secresomes at higher levels as compared to E7046. The six compounds were added to the culture medium at a concentration from 5 μg/mL to 75 μg/mL. E7046 was added at 10 μg/mL. The compounds of this invention induced release of extracellular vesicles at a level in a range from 100,000 to 500,000 (e.g., 450,000) EV/μL and proteins at a level from 0.18 to 0.7 μg/μL.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their activities to induce release of secresomes. Thus, other embodiments are also within the claims.

What is claimed is:

1. A compound of Formula (I): 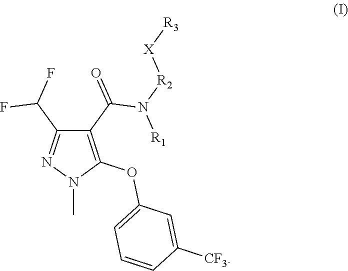
in which
R₁ is H or NH₂, or R₁ and R₂, together with the nitrogen atom they bond to, form a heterocycloalkyl ring;
R₂ is a bond, CH₂, (CH₂)₂, (CH₂)₃, (CH₂)₄, (CH₂)₅, (CH₂)₄CH(COOH), or (CH₂)₆;
X is a bond, C(═NH), O, NH, NR³′, N(NH₂), NHC(O), or NHC(S), R^{3′} being C₁-C₆ alkyl; and
R₃ is phosphonate, phosphonic, carboxylic, C₃-C₆ alkyl, C₁-C₆ alkoxy, C₃-C₁₀ cycloalkyl, 3- to 10-membered heterocycloalkyl, amino, aryl, or heteroaryl.

2. The compound of claim 1, wherein R₁ is H; each of R₂ and X is a bond; R₃ is aryl, heteroaryl, cycloalkyl, or heterocycloalkyl; and each of aryl, heteroaryl, cycloalkyl, and heterocycloalkyl is substituted or unsubstituted.

3. The compound of claim 2, wherein R₃ is aryl or heteroaryl substituted with heterocycloalkyl, heteroaryl, amido, trifluoromethyl, alkoxy, carboxylate, or carboxamide.

4. The compound of claim 2, wherein R₃ is

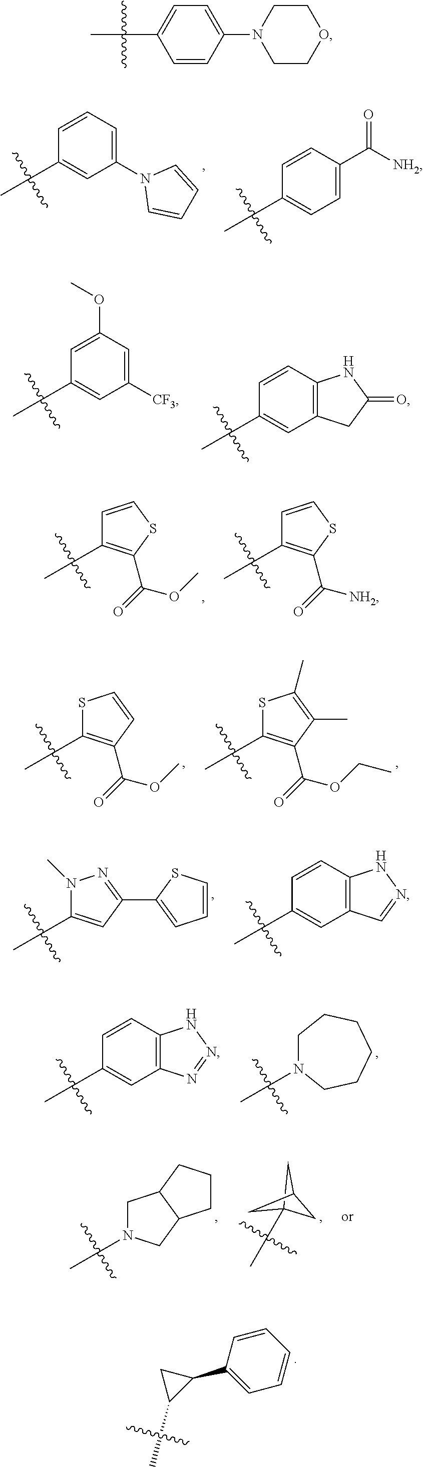

5. The compound of claim 1, wherein R₁ is H; R₂ is a bond, CH₂, (CH₂)₂, (CH₂)₃, (CH₂)₄, (CH₂)₅, or (CH₂)₆;
X is a bond; and
R₃ is aryl, heteroaryl, alkoxy, cycloalkyl, heterocycloalkyl, amino, phosphonate, or C2-C₆ alcohol.

6. The compound of claim 5, wherein R₃ is unsubstituted or substituted with halo, OH, NH₂, alkyl, carboxylate, alkoxy, sulfonamide, or carboxyl.

7. The compound of claim 6, wherein R₂—X—R₃ is

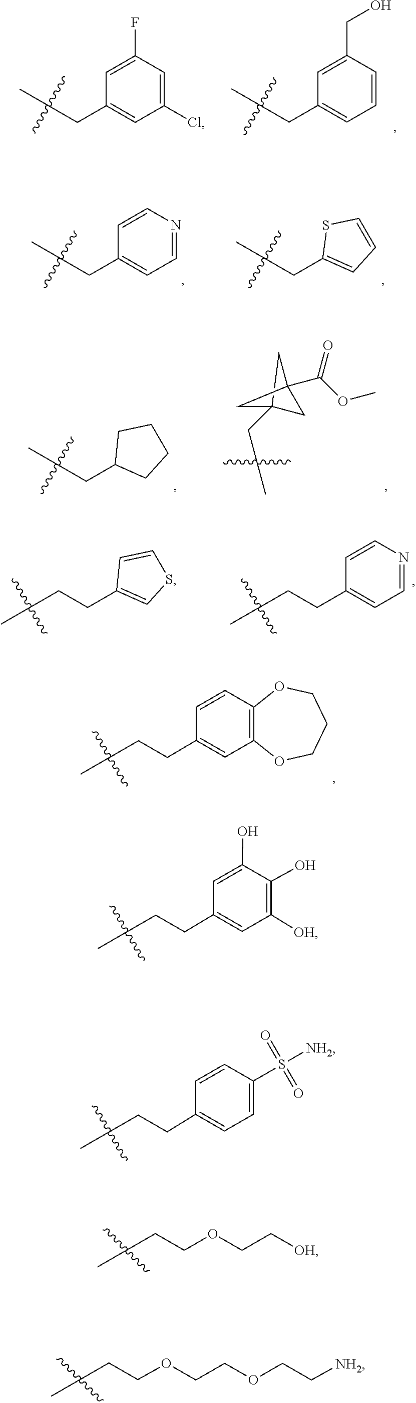

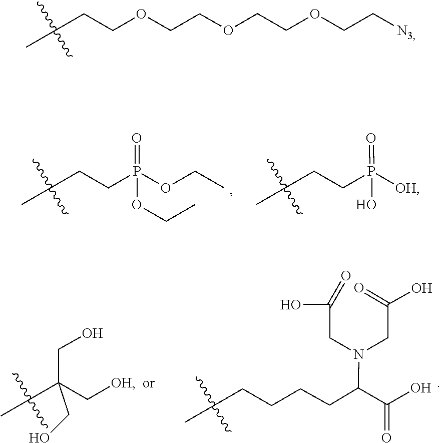

8. The compound of claim 1, wherein R₁ is H; R₂ is a bond; X is NH, N(NH₂), NHC(O), or NHC(S); and R₃ is amino, C₃-C₆ alkyl, C₁-C₆ alkoxy, C₃-C₁₀ cycloalkyl, 3- to 10-membered heterocycloalkyl, aryl, or heteroaryl.

9. The compound of claim 8, wherein X—R₃ is

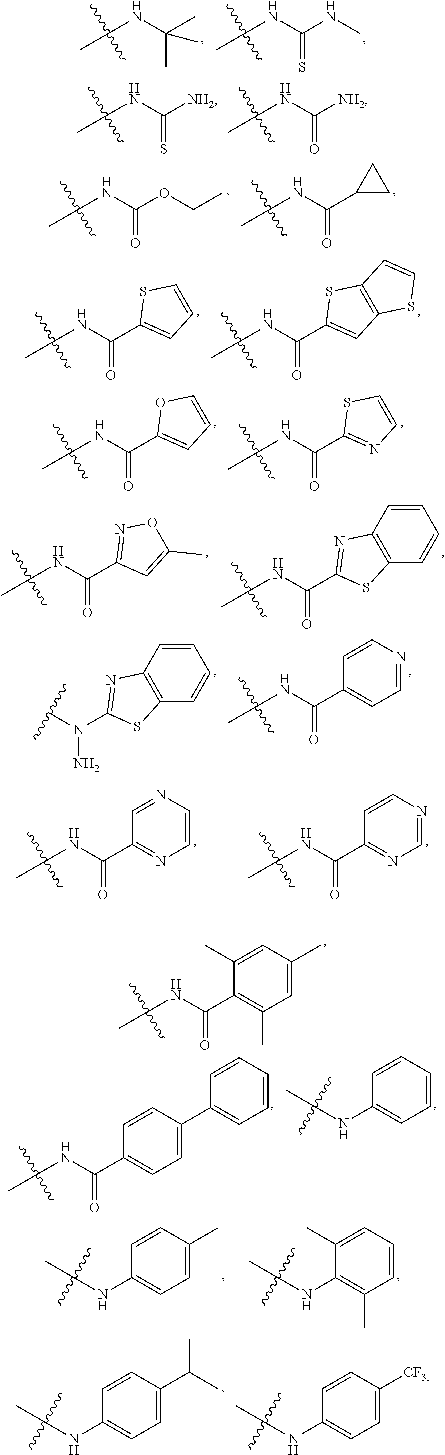

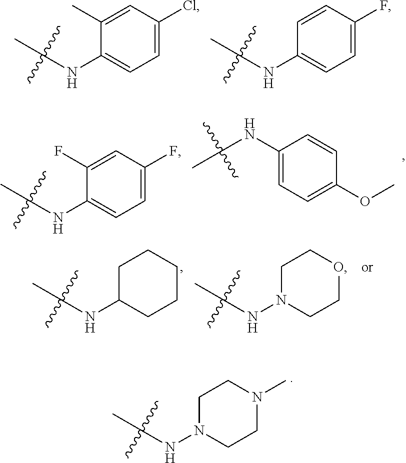

10. The compound of claim 1, wherein R₁ and R₂, together with the nitrogen atom they bond to, form a heterocycloalkyl ring;
X is a bond, C(═NH), NH, N(NH₂), NHC(O), or NHC(S); and
R₃ is amino, C₃-C₆ alkyl, C₃-C₁₀ cycloalkyl, 3- to 10-membered heterocycloalkyl, aryl, or heteroaryl.

11. The compound of claim 10, wherein N(R¹)—R₂—X—R₃ is

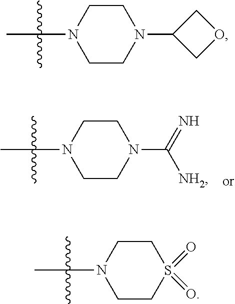 [SMILES: *N1CCN(C2COC2)CC1 ; *N1CCN(C(=N)N)CC1 ; *N1CCS(=O)(=O)CC1]

12. The compound of claim 1, wherein the compound is selected from the group consisting of Compounds 1-67.

13. The compound of claim 1, wherein the compound is selected from the group consisting of Compounds 8, 12, 28, 45, 54, and 66.

14. A method of producing secresomes enriched for proteins or mRNAs, the method comprising:
obtaining an isolated population of stem cells, wherein the stem cells are mesenchymal stem cells, mammary epithelial stem cells, neural stem cells or cancer stem cells,
culturing the isolated population of stem cells in a culture medium suitable for culturing the stem cells, wherein the culture medium contains an amount of a compound of claim 1, the amount being effective for inducing release of secresomes enriched for proteins and miRNAs for maintenance of stem cell properties or for cell regeneration from the stem cells, for a sufficient period of time to allow release of the secresomes, whereby the secresomes are released from the stem cells into the culture medium, and isolating the secresomes from the culture medium, and have a higher protein and miRNA content for maintenance of stem cell properties or for cell regeneration, as compared to control stem cells not treated with compounds.

15. The method of claim 14, wherein the stem cells are cultured for 4 to 8 days.

16. The method of claim 14, wherein the effective amount of the compound of claim 1 is 1-40 μg/mL.

17. The method of claim 14, wherein the stem cells are mesenchymal stem cells, mammary epithelial stem cells, or neural stem cells.

18. The method of claim 14, wherein the isolating step includes collecting the culture medium containing the secresomes released from the stem cells.
